(19) United States
(12) Patent Application Publication
(10) Pub. No.: US 2006/0259063 A1
(43) Pub. Date: Nov. 16, 2006

(54) Wire guides having distal anchoring devices

(76) Inventors: Brian L. Bates, Bloomington, IN (US); David V. Gorky, Flemington, NJ (US); Mark A. Lorenz, Peru, IN (US)

Correspondence Address: BRINKS HOFER GILSON & LIONE/CHICAGO/COOK, PO BOX 10395, CHICAGO, IL, 60610, US

(21) Appl. No.: 11/410,723

(22) Filed: Apr. 25, 2006

Related U.S. Application Data
(60) Provisional application No. 60/674,541, filed on Apr. 25, 2005.

(51) Int. Cl. A61M 29/00

(52) U.S. Cl. 606/198

(57) ABSTRACT

Improved wire guide devices having distal anchoring devices and methods of them percutaneously are provided. One device includes a wire guide having a distal portion operatively coupled to a holding body having a self-expanding end portion having wire members capable of assuming a first radially compressed configuration and a second radially expanded resilient configuration. Another device includes a wire guide distal portion operatively coupled to a distal anchoring device having a distal self-expanding suspension portion having a plurality of stabilizers capable of assuming a first radially compressed configuration when constrained by the outer sheath and a second radially expanded resilient configuration when the sheath is withdrawn proximally. An elongate outer sheath with first and second openings defining a lumen therebetween and slideably constrain the self-expanding portions to the compressed configuration. Expanded configurations help keep the wire guide distal portion at a target site within a body lumen.

### RELATED APPLICATION

The present patent document claims the benefit of the filing date under 35 U.S.C. §119(e) of U.S. Provisional Patent Application filed on Apr. 25, 2005 entitled, “Improved Wire Guides Having Distal Anchoring Devices,” and having an application Ser. No. 60/674,541, the disclosure of which is hereby incorporated by reference in its entirety.

### FIELD OF THE INVENTION

The present invention relates to medical devices and more particularly to wire guide anchoring devices for percutaneous use with interventional cardiology and other intra-luminal procedures, such as peripheral percutaneous transluminal angioplasty, and methods of using those devices.

### BACKGROUND OF THE INVENTION

Percutaneous transluminal angioplasty (“PTA”) provides an alternative treatment procedure for maintaining vessel patency when the body lumen (e.g., vessel passageway), which may have become partially narrowed or occluded, needs reinforcement, support, repair, or otherwise improved performance to restore or increase blood flow through the diseased section of the vessel. The terms “body lumen,” “passageway,” and “vessel passageway” include any bore, cavity, chamber, channel, duct, flow passage, lumen, opening, or orifice for the conveyance, regulation, flow, or movement of bodily fluids and/or gases of an animal. To cite but a few examples of vessels, PTA may be considered for use in dilating lesions, for instance, located in the body lumens of peripheral, renal, and coronary arteries, as well as the arterial and venal vascular system, aorta, colon, esophagogastrointestinal tract, pulmonary system, and other locations in a human or animal body (collectively, “vessel” or “body”) to name a few. Consequently, the PTA technique provides a physician, operator, or other healthcare professional (“physician”) with a minimally invasive non-surgical choice of treatment or investigation of a vessel in lieu of, for instance, open vascular surgery and other less conservative procedures.

Physicians may consider using PTA techniques with balloon angioplasty. In addition to simple balloon angioplasty, various atherectomy devices for removing plaque causing vessel stenosis have expanded the uses for PTA. These plaque removing devices include excimer laser angioplasty devices for photoablation of plaque, rotational atherectomy for mechanically ablating plaque, and directional atherectomy for cutting out plaque. As an adjunct to angioplasty, techniques involving intravascular, coronary, and vessel delivery devices for use with self-expanding devices such as stents, prosthetic valve devices, and other implantable articles to be referred to hereafter collectively as “stents,” help to prevent vessel wall collapse. For positioning the angioplasty device, stent delivery devices, and other diagnostic and treatment instruments, tools, and catheter delivery devices (individually and collectively, “PTA devices”), PTA techniques commonly employ placement hardware.

A wire guide typically serves as the placement hardware for delivering PTA devices into a body lumen percutaneously. A physician may use a cannula or a needle as a way of introducing the wire guide. For instance, the physician may create an incision in the patient and then position the cannula in the incision for inserting the wire guide.

The typical wire guide has an elongate (long) body with proximal and distal ends and tapers distally. As is conventional, “distal” means the end that is directed or oriented away from the physician when the device is inserted into a patient while “proximal” means the end that is closer to or toward the physician relative to the distal end. The terms “tapering,” “taper,” “tapered,” “tapers,” and variations thereof comprise a decreased, reduced, lesser, and/or smaller cross sectional area, mean diameter, perimeter, volume over a given length, thickness in height and width, and/or other smaller configuration, shape, form, profile, structure, external outline, and/or contour. Thus, the diameter decreases along the length of the wire guide, e.g., the distal end in one embodiment has a smaller effective outer diameter relative to the proximal end. A physician inserts the narrower distal end into a proximal end of the cannula or needle and then out a distal end of the cannula or needle, and thus into the body lumen. Once inside the body lumen, the wire guide may be advanced and manipulated until the distal end of the wire guide reaches its destination or target site. Alternatively, the physician may first place the wire guide into any one of a variety of vessels, such as an artery, bile duct, brachial vein, cephalic vein, or other vessel as described above, and then may introduce the cannula or needle over the wire guide and into the vessel. In subsequent steps, the physician may withdraw the needle over the wire guide and introduce a guide catheter over the wire guide and into the patient.

Regardless of how the physician places the wire guide into a body lumen for use with a traditional PTA device, the physician must ensure to position the wire guide properly. Put differently, the physician places the wire guide distal end at or near the target site for treatment, diagnosis, investigation, or medical intervention. As wire guide diameters at the distal end become smaller and smaller, however, physicians encounter a challenge to keeping the wire guide distal end properly positioned at the target site, particularly when other devices are advanced or withdrawn over the wire guide.

Different types of vessels add to the difficulty of holding the wire guide in place. In contrast to smaller caliber body lumens, vessels having larger lumens loosely constrain the wire guide distal end, especially considering that the wire guide tapers distally to a small effective outer diameter at the target site. Also, vessels having short take-offs present a challenge to holding the wire guide distal end in place. As but one example, renal arteries have a short take-off, and the wire guide distal end tends to withdraw proximal to the lesion and thereby relocate in the aorta.

Wire guide movement or migration results in the need for the physician to reposition the wire guide and return it to the desired placement at the target site. Repositioning the wire guide may prove difficult. Even under the best conditions, repositioning procedures may be time consuming.

Despite advances in PTA devices, the placement hardware has not kept up with the need to maintain the wire guide in position. Therefore, improved wire guides would be desirable. As taught herein, these wire guides can be placed and anchored at the desired position, which saves time for the physician and patient during the PTA procedure, and improves the quality of healthcare.

### SUMMARY OF THE INVENTION

Medical devices for percutaneous uses are provided. One embodiment comprises a wire guide having a distal portion operatively coupled to a holding body. The holding body has a self-expanding portion capable of being compressed to a first radially compressed configuration when constrained and capable of being expanded to a second radially expanded resilient configuration for engaging an inner wall of a body lumen for resisting migration of the wire guide distal portion.

In another embodiment of a wired guide device for deployment in a body lumen, the device includes an elongate outer sheath having a first opening and a second opening defining a sheath lumen therebetween. The device also includes a wire guide having a distal portion slideably received in the sheath lumen, and a wire guide anchor device operatively coupled to the wire guide distal portion, wherein the wire guide anchor device has a distal self-expanding suspension portion capable of being compressed to a first radially compressed configuration when constrained and capable of being expanded to a second radially expanded resilient configuration for engaging an inner wall of a body lumen for resisting migration of the wire guide distal portion.

Methods of providing a wire guide for percutaneous procedures on a patient are also provided. In one embodiment, a wire guide anchoring system is provided having an outer sheath with a distal first end portion, a proximal second end portion, and an elongate flexible middle section, the outer sheath further having a first opening and a second opening defining a lumen, the lumen being sized to slideably receive a wire guide. The wire guide has a proximal portion, an elongate flexible intermediate section, and a distal portion that secures a self-expanding anchor device that is capable of assuming a first radially compressed configuration when constrained by the outer sheath and a second radially expanded resilient configuration that engages an inner wall of a body lumen when the outer sheath is withdrawn proximally relative to the self-expanding anchor device. At a target site in said patient, the outer sheath distal first end portion is positioned with the wire guide distal portion and self-expanding anchor device within the outer sheath lumen, the self-expanding anchor device being in the first radially compressed configuration. The outer sheath is withdrawn from the wire guide distal portion such that the self-expanding anchor device expands to the second radially expanded resilient configuration for engaging the inner wall of a body lumen for migration resistance.

### BRIEF DESCRIPTION OF THE DRAWINGS

FIG. 1 is a schematic view, broken away, according to one embodiment of the invention.

FIG. 2 is a schematic view, broken away, according to an alternative embodiment of the invention.

FIG. 3 provides a longitudinal side view, broken away and partially sectioned, of a distal portion of an embodiment of a wire guide according to the invention.

FIG. 4 provides a longitudinal side view, broken away and partially sectioned, of a distal portion of an alternative embodiment of a wire guide according to the invention.

FIG. 5A is a schematic drawing of a convex wire member according to one embodiment of the invention.

FIG. 5B is a perspective view of a concave wire member according to one embodiment of the invention.

FIG. 5C is a perspective view of a radially expanded concave wire guide holding body self-expanding end portion according to one embodiment of the invention.

FIG. 5D is a perspective view of a radially expanded convex wire guide holding body self-expanding end portion according to one embodiment of the invention.

FIG. 6 provides a schematic view of a wire guide anchoring system having a holding body according to an embodiment of the invention.

FIG. 6A shows an enlarged partial view, broken away, of embodiments of a self-expanding end portion having an anchor barb according to the invention.

FIG. 6B shows an embodiment of FIG. 6A engaging a vessel wall.

FIG. 6C shows an enlarged partial view, broken away, of an alternative embodiment of a self-expanding end portion having an anchor barb engaging a vessel wall according to the invention.

FIG. 6D shows an enlarged partial view, broken away, of another embodiment of a self-expanding end portion having an anchor barb engaging a vessel wall according to the invention.

FIG. 7 provides a schematic view of a wire guide anchoring system having a holding body according to an alternative embodiment of the invention.

FIGS. 8 through 10 provide schematic views, broken away, of additional alternative embodiments of a holding body according to an embodiment of the invention.

FIG. 11 provides a schematic view, broken away, of a wire guide anchor device having a distal self-expanding suspension portion according to an alternative embodiment of the invention.

FIG. 11A is an end view of FIG. 11.

FIG. 12 is a block diagram illustrating a method of providing a wire guide anchoring system for holding a wire guide in place at a desired position.

FIG. 13 is a block diagram illustrating an alternative method of providing a wire guide anchoring system for holding a wire guide in place at a desired position.

### DETAILED DESCRIPTION OF THE PREFERRED EMBODIMENTS

Although not limited in its scope or applicability, the present invention relates generally to medical devices used percutaneously, as with peripheral, renal, and coronary arteries, as well as the aorta, pulmonary system, arterial and venal vascular system, esophagogastrointestinal tract, colon and other locations in a human and, more particularly, a body lumen. More particularly and by way of illustration and not by way of limitation, the present invention relates to wire guides having anchor devices for maintaining the wire guide in the desired position at the target site.

For the purpose of promoting an understanding of the principles of the present invention, the following provides a detailed description of embodiments of the invention as illustrated by the drawings as well as the language used herein to describe aspects of the invention. The description is not intended to limit the invention in any manner, but rather serves to enable those skilled in the art to make and use the invention. As used herein, the terms comprise(s), include(s), having, has, with, contain(s) and variants thereof are intended to be open ended transitional phrases, terms, or words that do not preclude the possibility of additional steps or structure.

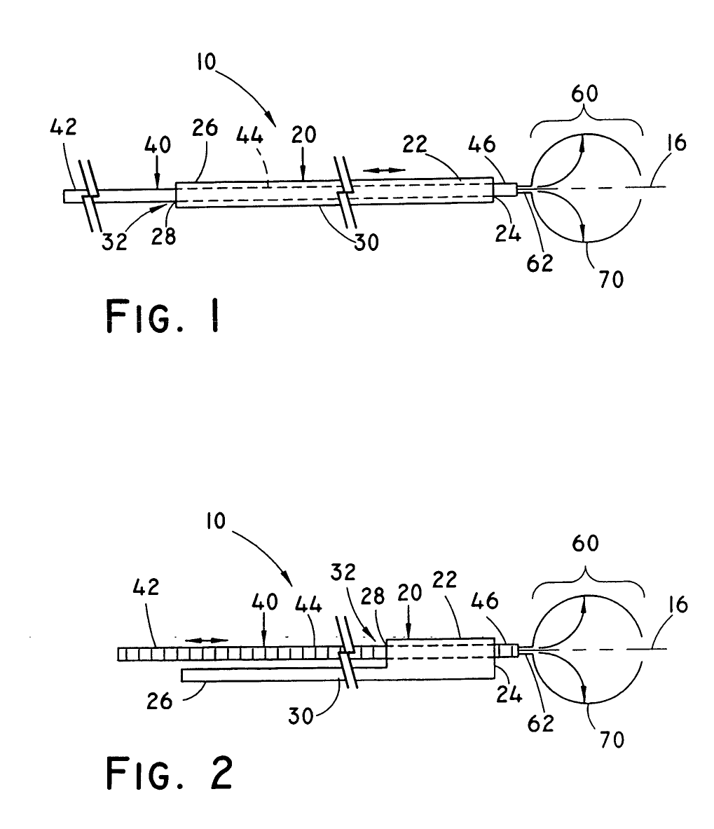

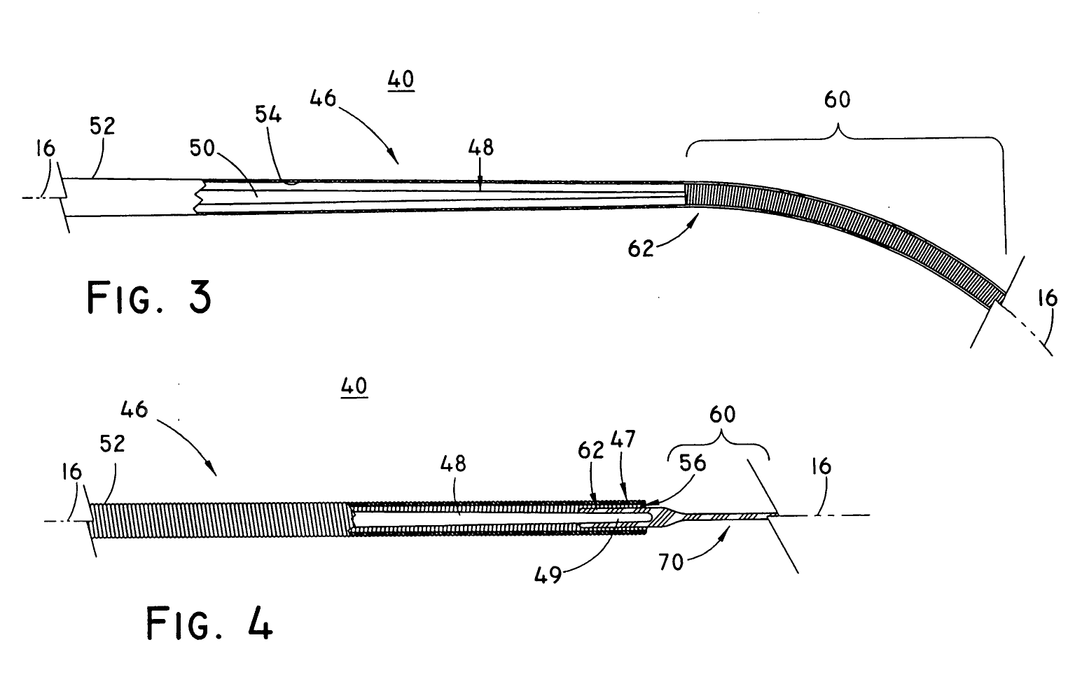

FIGS. 1 and 2 provide alternative embodiments of an improved wire guide anchoring system 10 according to the invention. The system 10 includes an outer sheath 20 configured for slideably receiving a wire guide 40 configured to be slideably received in the outer sheath 20, and a holding body 60 comprising super-elastic memory metal alloy secured to the wire guide 40 but not to the outer sheath 20. These figures show the outer sheath 20 partially withdrawn proximally relative to the wire guide 40 such that a holding body self-expanding end portion 70 has been allowed to return to its original elastic memory shape and thereby assume a radially expanded resilient configuration relative to a longitudinal axis 16. The term “longitudinal axis” as used herein and throughout to describe embodiments of the invention should be considered to be the approximate lengthwise axis. The longitudinal axis 16, which could be straight, may at other times be curved because portions of the wire guide 40, a wire guide distal portion 46 for example, may be at least partially flexible and, thus, may bend. Likewise, the holding body self-expanding end portion 70 is flexible and may bend away from the longitudinal axis 16.

Outer Sheath

The wire guide anchoring system 10 employs an elongate (long) sheath 20 having a distal first end portion 22, a proximal second end portion 26, and an elongate flexible middle section 30. Like the middle section 30, the first end portion 22 may also be sufficiently flexible to permit navigation of a tortuous vessel passageway and/or body lumen and to avoid damaging the vessel through which the physician advances the outer sheath 20, wherein vessel passageway and body lumen are used interchangeably herein and throughout. Sheaths of this type are available from Cook Incorporated, of Bloomington, Ind.

In addition, a first opening 24 and a second opening 28 define a wire guide lumen 32 therebetween. As used herein to describe embodiments of the invention, the term “wire guide lumen” includes any aperture, bore, cavity, chamber, channel, flow passage, opening, orifice, or passageway sized for slideably receiving at least a portion of the wire guide 40. The first opening 24 generally is disposed at or near the first end portion 22. Although the second opening 28 may be disposed at or near the second end portion 26 in the case of a delivery system commonly referred to as an “over-the-wire” delivery system and illustrated schematically in FIG. 1, the second opening 28 may also be positioned at or near the first end portion 22 but proximal to the first opening 24 as in the case of a rapid exchange type delivery system as illustrated schematically in FIG. 2. The phrase “at or near” as used to describe an embodiment of the invention includes a location that is at or within a relatively short distance of the most distal tip of the first end portion 22 such as, for instance, from about 0.5 centimeters (“cm”) to about 40 cm in length, although the range could also from about 0.1 cm to about 15.0 cm, as well as other suitable ranges as desired. Also, the first opening 24 may be positioned on the sidewall of the first end portion 22.

Embodiments of the outer sheath 20 include a tubular structure such as an elongate catheter, introducer, cover, shroud, case, or other tubular delivery device having a lumen 32 configured for slideably receiving the wire guide 40. The outer sheath 20 also includes a catheter, a working channel of an endoscope, and an endoscope accessory device having a channel for slideably receiving a self-expanding end portion of a holding body or a distal self-expanding suspension portion of a wire guide anchor device according to embodiments of the invention described below and thereby constraining the self-expanding end portion to a first radially compressed configuration and moving proximally over the self-expanding end portion in order to allow the self-expanding end portion to expand to a second radially expanded resilient configuration that engages a body lumen.

As used here to describe embodiments of the invention and not as any lexicographic definition, “tubular” includes any approximately general tube-like, cylindrical, shaft-like, rounded, oblong, elongated structure or member having a first opening 24 and a second opening 28 spaced proximal the first opening 24 and defining a wire guide lumen 32 therebetween. In one embodiment, the lumen 32 is sized to receive a wire guide 40 ranging in diameter from about 0.40 millimeters (“mm”) to about 1.00 mm, although the lumen may be larger or smaller according to the wire guide size intended for use with the outer sheath 20. Furthermore, given the configuration of vessels, vessel passageways, and/or body lumens, an outer sheath first end portion 22 that is generally tapered, rounded, or chamfered may be better tolerated by the patient. Further, in certain embodiments, the first end portion 22 may be soft and flexible so as to provide further protection for and care to the patient.

The outer sheath 20 may be constructed to have any outer diameter and length required to fulfill its intended purposes. The overall length of the outer sheath 20 may vary between about 40 cm and about 300 cm in length, although the sheath may be shorter or longer, as desired for reaching the target site within the body. The outer diameter may vary, too, and those diameters may decrease in the distal direction of the outer sheath 20, because the outer sheath optionally may taper in the distal direction at or near the first end portion 22. For instance, the diameters may vary between about 0.25 mm and about 1.25 mm, although the diameter may be greater or lesser than this range at certain positions along the length of the outer sheath 20. For instance, the diameter of the outer sheath 20 may be greater than 1.25 mm at the second end portion 26 and less than 0.25 mm at the first end portion 22. The outer sheath 20 may vary along its length in French size, too. For example, the outer sheath 20 may range in size from approximately 4.0 French to approximately 8.2 French.

The outer sheath 20 may be made of any suitable material (natural, synthetic, plastic, rubber, metal, composite, or combination thereof). Thus, in general, the material may comprise a synthetic material that may include, by way of example only and not by way of limitation, polyurethane, cellulose acetate, cellulose nitrate, silicone, polyethylene teraphthalate, polyamide, polyether block amide, polyester, polyorthoester, polyanhydride, polyether sulfone, polycarbonate, polypropylene, high molecular weight polyethylene, polytetrafluoroethylene, or mixtures or copolymers thereof, polylactic acid, polyglycolic acid or copolymers thereof, a polyanhydride, polycaprolactone, polyhydroxy-butyrate valerate, polyhydroxyalkanoate, or another polymer or suitable material. Further, the material for the outer sheath 20 may be biocompatible or capable of being made biocompatible, such as by coating, chemical treatment, or the like.

Wire Guide

Furthermore, the wire guide anchoring system 10 uses a wire guide 40 comprising a proximal portion 42, an elongate flexible intermediate section 44, a distal portion 46, and a holding body 60 secured to the distal portion 46, although the wire guide anchoring system 10 does not preclude other features. The term “intermediate” in describing an embodiment of the wire guide intermediate section 44 is intended to mean between, though not necessarily equidistant to, the distal tip of the wire guide distal portion 46 and the proximal tip of the wire guide proximal portion 42. Like the intermediate section 44, the wire guide distal portion 46 may also be sufficiently flexible to navigate the tortuous vessel passageway and slideably move within the outer sheath 20. Also, given the configuration of body lumens, vessels, and vessel passageways to be navigated, the wire guides may have a wire guide proximal portion 42, wire guide intermediate section 44, and wire guide distal portion 46 that are generally tubular. In order to increase flexibility and thereby reduce the risk of damaging a vessel passageway, the wire guide typically tapers distally to smaller cross-sections.

The wire guide 40 is sized so that the wire guide distal portion 46 and at least some of the wire guide intermediate section 44 are slideably received in the sheath lumen 32, and the overall length of the wire guide 40 may be longer or shorter than the length of the outer sheath 20. For instance, in the case of an “over-the-wire” delivery system, the wire guide 40 may need to be slightly more than twice as long as the sheath or other tools, such as replacement or exchange catheters, balloon catheter devices, stent delivery devices, prosthetic valve delivery devices, angioplasty devices, atherectomy devices, and other medical devices (collectively, “over-the-wire guide instruments”) intended for placement over the wire guide 40 and inside a patient's body. For instance, if reaching the target site requires an outer sheath 20 measuring about 60 cm in length, then a wire guide 40 in a conventional over-the-wire guide instrument may need to be slightly longer than approximately 120 cm in length, because the physician will need to hold or secure the wire guide proximal portion 42 (i.e., the portion extending out of the body) as the physician loads the outer sheath 20 onto or removes the outer sheath 20 from the wire guide 40. Likewise, replacement, exchange, or use of over-the-wire guide instruments requires that a physician secure the wire guide proximal portion 42 of this lengthy wire guide 40 while the outer sheath 20 is withdrawn from the patient until the outer sheath first end portion 22 is then slipped off of the wire guide proximal portion 42. In a rapid exchange delivery system, the wire guide 40 need only be slightly longer than the outer sheath 20 so that, when the outer sheath lumen 32 receives at least a portion of the wire guide 40 and the physician positions the device within the patient's body, the wire guide proximal portion 42 extends out of the patient and proximally of the sheath second end portion 26 by a short distance equal to the length of the sheath lumen 32.

Accordingly, the wire guide 40 may have a length of two ranges. In a conventional rapid exchange system, the wire guide 40 measures from slightly more than about 40 cm to slightly more than about 300 cm in length, whereas the wire guide 40 may be twice that length in an over-the-wire guide delivery system. The wire guide 40 may be shorter or longer than these two ranges, as desired for reaching an intended target site within the body. In general, the wire guide 40 may have a length from approximately 40 cm to approximately 300 cm in length, although it may be more or less than this range as desired for the intended treatment site within the patient.

The wire guide outer diameter may vary from one embodiment to the next, and may vary within the same embodiment along the length of the wire guide 40 if the wire guide tapers in the distal direction. In these embodiments, the outer diameter needs to be at least slightly less than the inner diameter of the lumen 32 of the outer sheath 20, so that the outer sheath 20 is slideable relative to at least a portion of the wire guide 40. In one embodiment, the wire guide diameter ranges from approximately 0.40 mm to approximately 1.00 mm. Similarly, a self-expanding end portion 70 of a holding body 60 or a distal self-expanding suspension portion 101 of a wire guide anchor device 100 according to embodiments of the invention described below needs to be compressible to a radially compressed configuration to be slideably received within the outer sheath lumen 32. If the wire guide 40, holding body 60, or wire guide anchor device 100 are nested or otherwise snug within the lumen 32, then they will tend to be pulled proximally when the physician withdraws the outer sheath 20 proximally. An outer sheath having an optional lubricious inner surface surrounding the lumen 32, such as an inner surface comprising a fluorocarbon such as polytetrafluoroethylene (PTFE), improves the slideability with a wire guide 40, holding body 60, or wire guide anchor device 100 sized to avoid a friction fit within the lumen 32. Likewise, the wire guide, holding body, or wire guide anchor device may have an optional slippery outer surface from inactive coatings, such as AQ® hydrophilic to provide a low coefficient of friction when wet.

The wire guide 42 may be made of any suitable material or combination of materials, wherein the wire guide distal portion 46 is sufficiently flexible and the intermediate section 44 may optionally be flexible. The material may need to be biocompatible or capable of being made biocompatible, such as by coating, chemical treatment, or the like. Thus, the wire guide distal portion 46, wire guide intermediate section 44, and/or wire guide proximal portion 42 may comprise any suitable material such as stainless steel, although they can be made from many other suitable materials. Other materials include biologically compatible metals, polymers, plastics, alloys (including super-elastic memory metal alloys), or composite materials that are either biocompatible or capable of being made biocompatible.

In one embodiment, the wire guide distal portion 46 and/or optionally the wire guide intermediate section 44 comprise a super-elastic memory metal alloy. Some examples of embodiments of super-elastic memory metal alloys include copper-zinc-aluminum, copper-aluminum-nickel, iron-manganese-silicon, gold-cadmium, copper-aluminum, copper-aluminum-nickel, cobalt-chromium-nickel-molybdenum-iron alloy, cobalt-chrome alloy, titanium, thermosetting polymers, or thermoplastic polymers and/or any combination thereof. In one embodiment, the super-elastic memory metal alloy is a nickel-titanium alloy (“nitinol,” an acronym of Nickel Titanium Naval Ordnance Laboratory, where the alloy's properties were discovered). Nitinol is a super-elastic memory metal alloy containing nearly equal numbers of nickel and titanium atoms, and the relative amounts of nickel and titanium can be varied by a few percent.

A super-elastic memory metal alloy is flexible and can accommodate some bending and twisting while still returning to its memory shape. The super-elastic memory metal alloy can be trained to take on a predetermined shape. These alloys exhibit this characteristic to greater or lesser degree. For example, nitinol is known for its flexibility and its low modulus, thereby allowing it to have low contact force and pressure while still having sufficient strength. Moreover, nitinol may be constructed to have a thermally triggered memory, such that it is manufactured below a temperature transformation level to a martensitic state in which case it is softened for loading in a catheter in a compressed and elongated state.

Super-elastic memory metal alloys have the desirable property of becoming rigid (e.g., returning to a remembered state), when heated above a transition temperature. When nitinol, for example, is heated above the transition temperature, the material undergoes a phase transformation from martensite to austenic, such that material returns to its remembered state. The transition temperature is dependent on the relative proportions of the alloying elements Ni and Ti and the optional inclusion of alloying additives. In one embodiment, nitinol regains its thermally triggered memory shape in an austenitic state when warmed to a selected temperature above the temperature transformation level, such as approximately 98.6 degrees Fahrenheit if the thermally triggered memory property is to be used at a body temperature for a human patient.

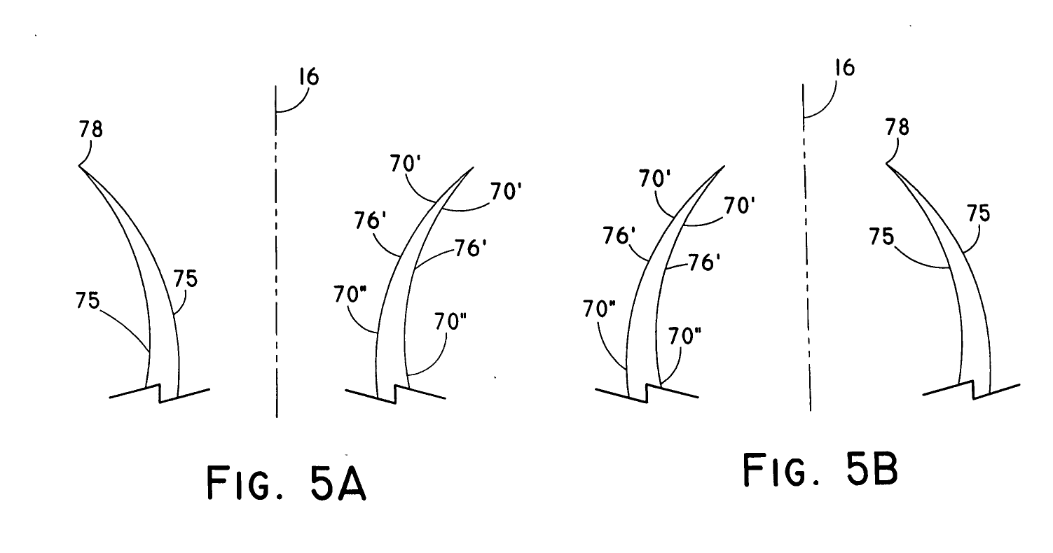

FIG. 3 illustrates an embodiment of a distal portion 46 of a wire guide 40 according to the invention. Wire guides of this type are available from Cook Incorporated, of Bloomington, Ind. The wire guide distal portion 46 has a flexible mandrel 48 running along an approximate center longitudinal axis 16 at the wire guide distal portion 46. The mandrel 48 may comprise stainless steel, nitinol, or any of the materials discussed above or combinations thereof. In addition, the mandrel 48 may taper (e.g., reduced diameter or cross sectional area) distally.

Furthermore, the wire guide 40 may include an optional covering 52, such as a coating or membrane, surrounding at least a portion of the mandrel 48, as shown in FIG. 3. In order to show the mandrel 48 inside the covering 52, FIG. 3 illustrates the covering 52 as cut away. The terms “covering,”“membrane,” “coat,” “coating,” “coated,” and variants thereof when used to describe any embodiment of the invention includes any substance, compound, molecule, or material (whether comprising a solid, liquid, fluid, gel, gas, or vapor) chemically bonded via covalent bonds, ionic bonds, or intermolecular bonds (such as ion-dipole forces, dipole-dipole forces, London dispersion forces, and/or hydrogen bonding), adhered, or otherwise applied by the method(s) of laminating, taping, dipping, spraying, depositing, vapor deposition, wrapping (thermally fusing together), painting and curing, and the like. The covering 52 may be of a generally uniform thickness or of different and/or varying thicknesses. The covering 52 may cover all of the outer surface of the wire guide 40 or some of the outer surface.

The covering 52 may be any suitable material, including but not limited to plastic, polymers, rubber, metal, alloys, composite, or combination that is biocompatible or capable of being made biocompatible, such as by coating, chemical treatment, or the like. The covering 52 may comprise a therapeutic agent, such as a drug, medication, narcotic, antibiotic, pharmaceutical product, and/or medicinal agent, therapy, or substance. Types of therapeutic agents may be active, such as medicine that is utilized during the medical procedure by, for example, assisting with the healing process, assisting to reduce bacterial count, and otherwise delivering medication. Specific examples of therapeutic agents include neomycin, sulfa drugs, antimicrobials, antibiotics, oxybutynin chloride, lidocaine, ketorolac, ketorolac tromethamine, ibuprofen, ketoprofen, Tylenol, and diclofenac and their equivalents, but these or solely for illustrative purposes and not by way of limitation. In one embodiment the therapeutic agent optionally may be composed to be soluble to provide timed or slow release.

In one embodiment, the covering 52 comprises an AQ® hydrophilic coated polymer to provide an optional slippery outer surface and low coefficient of friction when wet and, thereby, facilitating passage through stenosed or strictured anatomy. The covering 52 may comprise a Heparin coating, from a class of anticoagulants, or from a class of hydrophilic materials such as an AQ® hydrophilic coated polymer. The covering 52 may comprise the entire length of the wire guide 40, or in one embodiment, from about 10.0 cm to at least about 65.0 cm in length of the wire guide distal portion 46. The remaining proximal length of the covering 52 may comprise a fluorocarbon such as polytetrafluoroethylene (PTFE). In still another embodiment, the covering 52 is a hydrophilic material that includes a hydrogel (i.e., a polymer that typically is covalently bonded to the outer surface and is relatively dry until the physician applies water, at which time the polymer swells with an aqueous solution). A hydrogel commonly is 80-90%, and preferably between about 50-98% water by weight in equilibrium. Mechanically, a hydrogel is capable of supporting a tensile stress of between 40,000-60,000 dynes/cm2. Chemically, hydrogels tend to remain stable and not degrade in vivo.

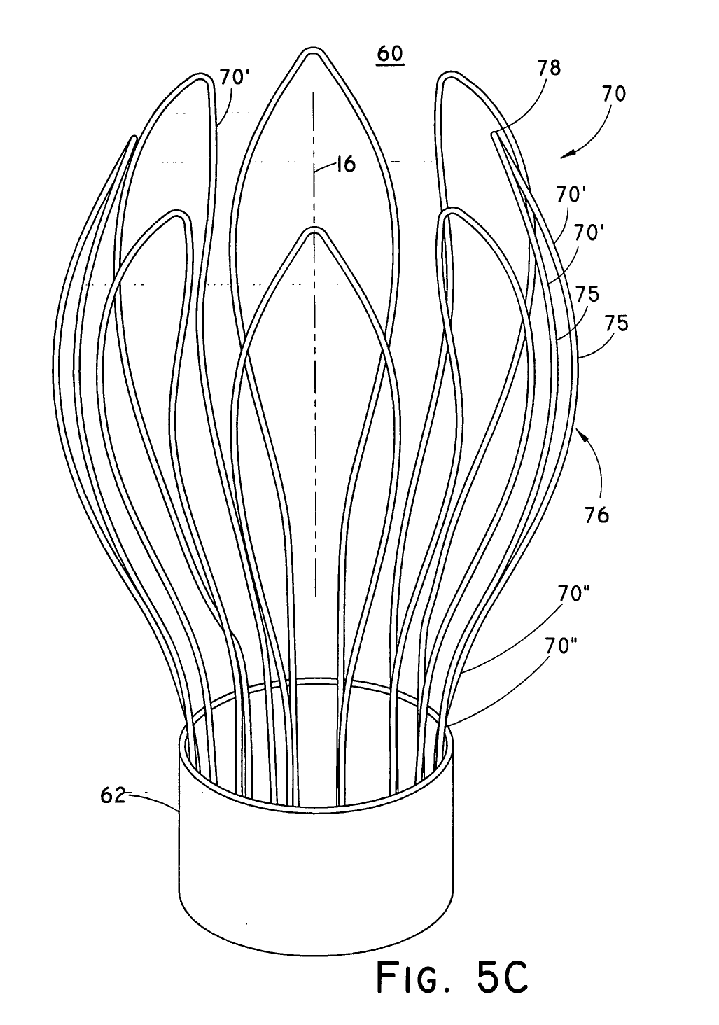

FIG. 4 provides an alternative embodiment of a distal portion 46 of a wire guide 40 according to the invention. The flexible mandrel 48 runs along an approximate center longitudinal axis at the wire guide distal portion 46, and the optional covering 52 is a coil or spring wound around the mandrel 48. In addition, the wire guide 40 has a mandrel distal end 49 tapered to form a cavity 56 at a wire guide distal end 47 of the wire guide distal portion 46. The cavity 56 is configured for receiving a mounting end portion 62 of the holding body 60 for operatively coupling the holding body 60 to the wire guide distal portion 46.

Holding Body and Wire Guide Anchor Device

The wire guide anchoring system 10 also comprises a holding body 60. Moreover, the holding body 60 comprises mounting end portion 62 and a self-expanding end portion 70, although the holding body 60 does not preclude other features. The self-expanding end portion 70 comprises any suitable super-elastic memory metal alloy or combination of super-elastic memory metal alloys, including but not limited to the super-elastic memory metal alloys described above. Stainless steel and super-elastic memory metal alloys such as nitinol are biocompatible and particularly shapeable, but super-elastic memory metal alloys such as nitinol have certain advantages for the self-expanding end portion 70.

For example, nitinol is less stiff than is stainless steel and, thereby, will not produce the same radial force in a similarly-sized wire formed of stainless steel. This renders the self-expanding end portion 70 (and the anchoring hooks discussed below) more atraumatic against an interior vessel wall. Moreover, nitinol, for example, may be cooled to transform the material to martensite, which is more ductile than austenite, making the self-expanding end portion 70 more malleable and more easily collapsible, thereby facilitating removable engagement the self-expanding end portion 70 against the interior vessel wall. Furthermore, super-elastic memory metal alloys such as nitinol in the collapsed state will be less prone to scrape, scratch, or tear the inner wall of the outer sheath 20, working channel of an endoscope, or other accessory device. Also, the actual ratio of expanded to collapsed size is a function of the material elasticity or how much deflection the material can absorb before being plastically deformed, and ANSI type 304 stainless steel might allow a 15 or 20% or degree deflection whereas nitinol in the super-elastic condition might allow a 30 to 40% or degree deflection.

In one embodiment, the holding body mounting end portion 62 may be stainless steel or other suitable metal or metal alloy. In another embodiment, the holding body mounting end portion 62 is a stainless steel, metal alloy, or super-elastic memory metal alloy filament or spring constrained by the covering 52. In yet another embodiment, the holding body mounting end portion 62 may be a safety wire fastened to the coil or spring covering 52. In still another embodiment, the holding body mounting end portion 62 may be integral with the mandrel 48.

The holding body mounting end portion 62 may be operatively coupled to the wire guide mandrel 48, the optional wire guide covering 52, or both. By way of example only and not by way of limitation, the terms “operatively coupling,” “operatively coupled,” “coupling,” “coupled,” and variants thereof are not used lexicographically but instead are used to describe embodiments of the invention having a point, position, region, section, area, volume, or configuration at which two or more things are mechanically, chemically, and/or chemical-mechanically bonded, joined, adjoined, connected, associated, united, mated, interlocked, conjoined, fastened, held together, clamped, crimped, friction fit, pinched, press fit tight, nested, wedged, and/or otherwise associated by a joint, a junction, a juncture, a seam, a union, a socket, a melt bond, glue, adhesives, resins, welding (laser, spot, etc.), soldering, brazing, adhesives, heat bonding, passive oxide layer covering, aluminum paste flux, chemical bonding materials, implanted arrangement, or combinations thereof.

For example, the mounting end portion 62 of the holding body 60 may be secured to the wire guide distal portion 46 by any suitable means, including but not limited to mechanical techniques such as crimping and swaging, or may be secured by heat bonding, glue, adhesives, resins, welding, soldering, brazing, adhesives, affixed by a passive oxide layer covering, aluminum paste flux, or the technique described in U.S. Pat. No. 5,354,623, the disclosure of which is incorporated herein by reference, or by chemical bonding materials or combinations thereof. In another embodiment, the mandrel 48 secures the holding body mounting end portion 62 and then receives the covering 52. The holding body mounting end portion 62 may lie between the mandrel outer surface 50 and the covering inner surface 54 for securing, connecting, attaching, adjoining, joining, or otherwise combining the mounting end portion 62 of the holding body 60 to the mandrel 48 of the wire guide 40. In another optional embodiment, the mandrel 48 is nested within the covering 52, and then the holding body 60 is secured to the outside of the covering 52. In these various embodiments, it should be understood that the holding body 60 may be secured by compression or friction fit between the mandrel outer surface 50 and the covering inner surface 54 and/or the mandrel 48 or covering 52.

As a result, the self-expanding end portion 70 is capable of assuming a first radially compressed configuration when constrained by the outer sheath 20 and a second radially expanded resilient configuration when not constrained by the outer sheath 20. When the outer sheath 20 is withdrawn proximally relative to the wire guide 40, the memory metal alloy returns to its elastic memory (i.e., a bended deflected state that curves or defects away from, or toward, the vessel wall) so as to assume a second radially expanded resilient configuration for substantially keeping the distal portion from moving proximally relative to the vessel passageway and relative to the proximal withdrawal of the outer sheath. As resilient is used herein and throughout, a second radially expanded resilient configuration describes embodiments that can be collapsed back to a first radially compressed configuration.

The self-expanding end portion 70 may comprise a wire, strand, and filament or a porous, nonporous, substantially semi-permeable, or impermeable membranous structure (individually and collectively, a “wire member 75”). In one embodiment, the self-expanding super-elastic memory-shaped alloy wire members 75 is round, but wire members 75 of any shape may be used, including rectangular wire, square wire, wedge or “pie-shaped” wire, flat wire and triangular wire. Each “wire” may comprise two or more wires twisted together for greater stiffness and control of the device. A wire member 75 may be formed by extrusion, or may be purchased in a form that is commercially available. Optionally, a wire member 75 may be turned or bent, shaped between two blocks with complementary curved interfaces corresponding to the desired shape, wrapped around, molded, and shaped onto a mandrel, or cut from a tube or sheet of material, laser cut, chemical etched, stamped, electric discharge machined, or formed by other known processes for manufacturing memory metal alloy. In one embodiment, the wire member 75 and/or the self-expanding end portion 70 has a thickness of at least about 0.012 inches.

In one embodiment, the wire member 75 comprises a wire having a round or near round cross-section with a diameter of at least 0.0125 inches. Of course, it is not necessary that the wire member 75 have a round cross-section. For example, the wire member 75 may have a curved transverse cross-section, such as, for example, a circular cross-section, or it may have a polygonal cross-section, such as, for example, a rectangular cross-section. Alternatively, the transverse cross-section of the wire member 75 may include both curved and straight portions.

When the self-expanding end portion 70 returns to a second radially expanded resilient configuration, the self-expanding end portion 70 optionally assumes J-shape, U-shape, S-Shape, V-shape, hoop-like, helical, curved, bent, angular, crescent shape, a spherical, cylindrical, elliptical, oval, umbrella, conical, oblong, tulip, umbrella shape, funnel shape, or basket shape so as to better conform to the vessel passageway when expanded. The invention is not limited to these shapes, however, and it should be understood as being of or relating to any structure that can be safely expanded so as to engage and hold against the inner walls of the body lumen.

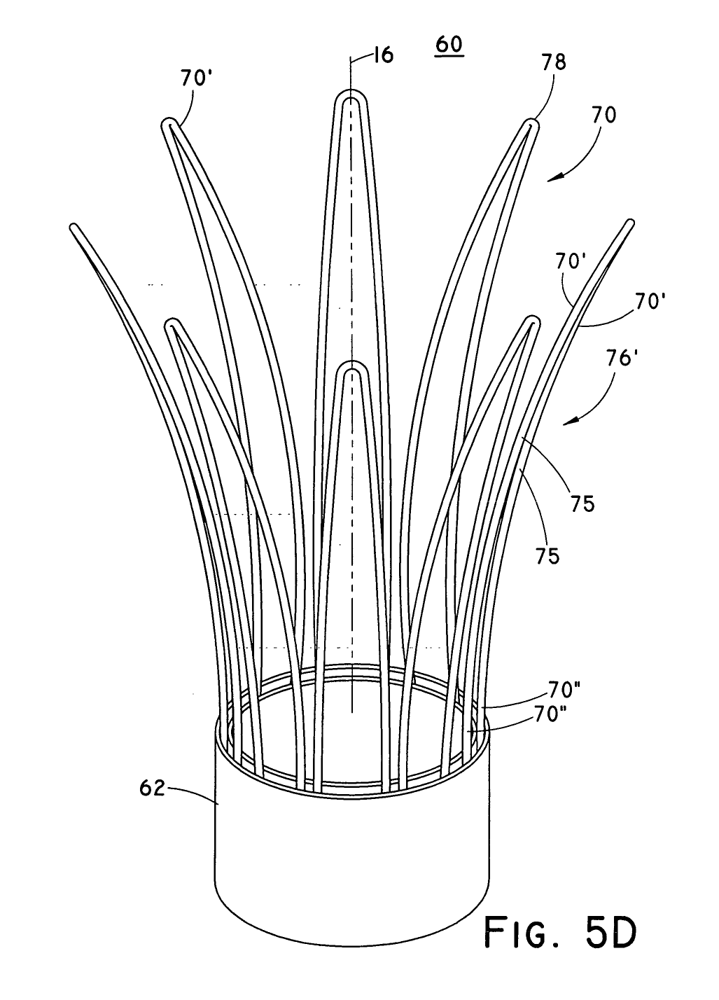

In the expanded configuration, the holding body self-expanding end portion 70 provides a sufficient radial force for holding the wire guide 40 in place so as to, for example, prevent inadvertent movement of the wire guide during exchange, avoid compromising blood flow, or to unintentionally damaging the vessel wall, thereby presenting less risk of accidental vessel injury. As one example only, if the body lumen is approximately 9 mm in diameter at the expanding site, then the holding body self-expanding end portion 70 may be manufactured to have a memory that expands effectively from about 9 mm to about 12 mm. In other words, the holding body self-expanding end portion 70 is capable of expanding to an effective outer diameter greater than the body lumen diameter. In describing the embodiments, effective outer diameter refers to a radial diameter and means a greatest diameter that the device is capable of expanding to (as will be understood there will be many diameters relative to a longitudinal axis of the self-expanding end portion 70). Embodiments of the self-expanding end portion 70, shown in FIGS. 1 and 2, will be further discussed with reference to FIGS. 5 through 11. In one embodiment, the self-expanding end portion 70 has a tensile strength of between about 285,000 pounds per square inch (psi) and 330,000 psi.

FIGS. 5A, 5B, 5C, and 5D show that the self-expanding end portion 70 may be concave or convex when disposed about the longitudinal axis 16 in a second radially expanded resilient configuration for engaging the vessel walls. In order to assist in explaining concave and convex as used in this specification to describe embodiments of the invention, and not as any lexicographic definition, the self-expanding end portion 70 may comprise a wire member 75 having a wire member distal end portion 70′ and a wire member proximal end portion 70″. In one embodiment according to the invention, adjacent wire members 75 are joined end-to-end by their wire member distal end portions 70′ at an arcuate connection 78 that curves from about 210 degrees to about 360 degrees from one wire member to an adjacent wire member, and in one embodiment the wire member distal end portion 70′ curves from about 270 degrees to about 330 degrees.

The arcuate connections 78 may be integrally formed from the adjacent wire member distal end portions 70′. In one embodiment, adjacent wire members 75 are formed from one wire turned or bent to form the arcuate connection 78, and the arcuate shape for the adjacent wire members 75 may be shaped between two blocks with complementary curved interfaces corresponding to the desired arcuate shape (e.g., concave or convex). In one embodiment, a plurality of pairs of adjacent wire members 75 joined by an arcuate connection 78 may be operatively coupled at their wire member proximal end portions 70″ to the mounting end portion 62, wherein the plurality of pairs form an arcuately shaped cylindrical self-expanding end portion 70. In an alternative embodiment, a pattern of adjacent wire members 75 and arcuate connection 78 may repeat by taking a length of a wire and wrapping it around, molding it, and shaping it onto a mandrel. In yet another embodiment, adjacent wire members 75 and arcuate connection 78 may be cut from a tube or sheet of material, laser cut, chemical etched, stamped, electric discharge machined, or formed by other known processes to form an arc-shaped cylindrical self-expanding end portion 70.

When adjacent wire members 75 are joined end-to-end by an arcuate connection 78 joining the adjacent wire member distal end portions 70′, the configuration of a first wire member 75-distal end portion 70-second wire member 75 in one embodiment may take on an approximate U-shape, V-shape, J-shape, Z-shape, zigzag shape, parabolic shape, serpentine, undulating, and the like in a radially expanded state. Because the arcuate connection 78 for joining the wire member distal end portions 70′ of adjacent wire members 75 end-to-end is curved, it has the benefit of atraumatically and releasably engaging a vessel wall without causing relatively serious tearing thereto, and may be compressed into a collapsed state without scraping, scratching, or damaging an outer sheath 20, working channel of an endoscope, or channel of an accessory for use with an endoscope. Also, the wire members 75 would be less prone to overlap or to become entangled, and this results in wire members 75 being able to expand radially apart.

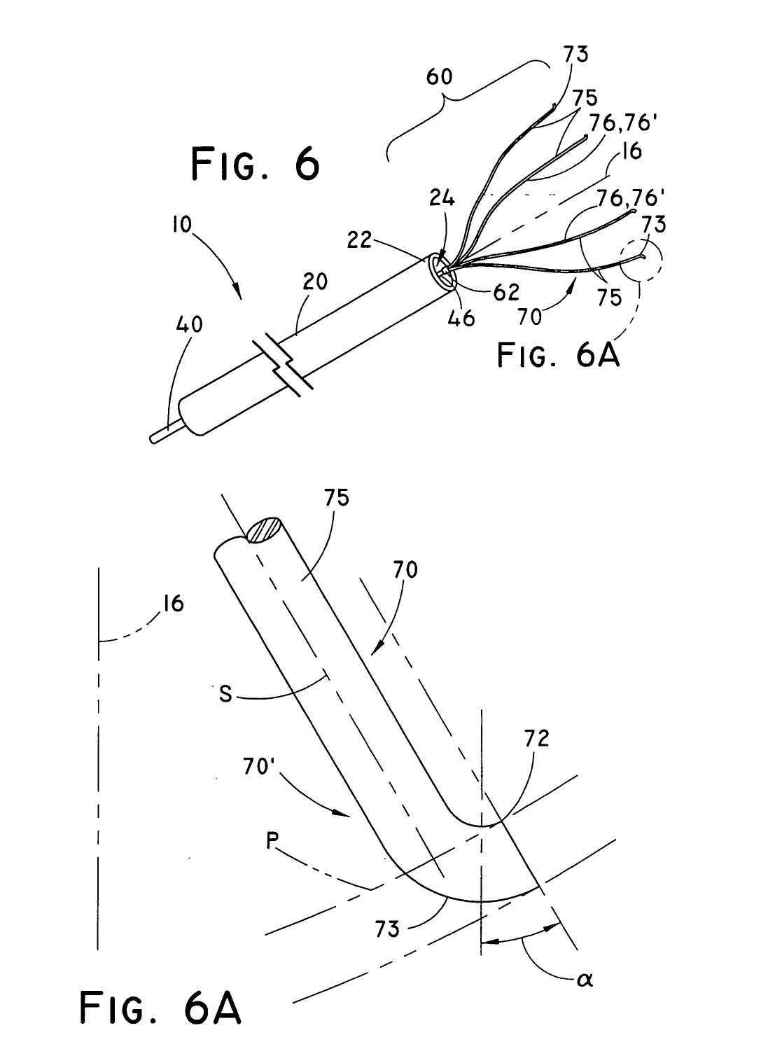

When adjacent wire members 75 are joined end-to-end at their wire member distal end portions 70′ by an arcuate connection 78 (e.g., FIGS. 5A-5D), or when a wire member 75 terminates at a distal end portion 70′ and/or an anchoring member (e.g., FIG. 6), the wire member 75 in the radially expanded state in one embodiment comprises an arc-shape intermediate the holding body mounting end portion 62 (and/or the wire member proximal end portion 70″) and the wire member distal end portion 70′ (and/or the arcuate connection 78). The arc-shaped wire members 75 may be either concave or convex when disposed about the longitudinal axis 16. In other words, the self-expanding end portion 70 may bend (i.e., arc) outwardly or inwardly of a cylindrical envelop-as opposed to being straight and lying on a cylindrical envelope. Concave and convex self-expanding end portions 70 provide additional unique results.

Referring to a self-expanding end portion 70 as concave 76 or convex 76′ is largely a matter of perspective as well as the frame of reference used in describing embodiments according to the invention. FIGS. 5A and 5B show, for example, enlarged schematic views of embodiments of self-expanding end portions 70, which optionally can be utilized in FIGS. 1, 2, 5C, and 5D in order to assist in explaining concave and convex as used in this specification to describe embodiments of the invention, and not as any lexicographic definition. As shown in FIGS. 5A and 5B, a self-expanding end portion 70 comprises a wire member 75 having a wire member distal end portion 70′ and a wire member proximal end portion 70″. The wire member 75 arcs (e.g., follows an arc-shaped course) intermediate the wire member distal end portion 70′ and a wire member proximal end portion 70″, and the arc may be either concave 76 or convex 76′. The longitudinal axis 16 helps to provide a frame of reference for labeling a self-expanding end portion 70 and/or a wire member 75 as either concave 76 or convex 76. When a plurality of self-expanding end portions 70 are disposed about the longitudinal axis 16 according to the invention, they are capable of engaging opposing vessel walls and thereby preventing the wire guide distal portion 46 from migrating within the body lumen.

For example, FIG. 5A depicts a wire member 75 that is convex 76′ in reference to the longitudinal axis 16 in the radially expanded state. In contrast, FIG. 5B depicts a wire member 75 that is concave 76 in reference to the longitudinal axis 16 in the radially expanded state. In other words, the wire member 75 bends (i.e., arcs) outwardly (e.g., concave 76) or inwardly (e.g., convex 76′) of a cylindrical envelop-as opposed to being straight and lying on a cylindrical envelope. In FIGS. 5A and 5B, the adjacent wire members 75 are joined end-to-end at their wire member distal end portions 70′ by an arcuate connection 78, but as explained above the adjacent wire members 75 may actually be formed from a single wire or even cut from a tube.

FIG. 5C shows an embodiment of the invention where there may be a plurality of wire guides 40 joined at a mounting end 62 and extending distally to self-expanding end portion 70. The mounting end 62 may be any structure, such as a metal cuff or collar, disposed about and operatively coupled by any means discussed above (e.g., heat bonding, glue, adhesives, resins, welding, soldering, brazing, adhesives, passive oxide layer covering, aluminum paste flux) to the prevent the wire guides from unraveling and otherwise separating prematurely at an undesired location along the longitudinal axis 16. The term “plurality,” as used to describe embodiments of the invention, means “two or more.” Thus, two or more wire guides 40 shall not preclude the possibility of additional wire guides 40 or additional features to the self-expanding end portion 70. The embodiments in the other figures showing a single wire guide 40 may also comprise a plurality of wire guides 40 for use with a vessel having a sufficiently sized vessel passageway (e.g., body lumen) for accommodating the increased diameter resulting from a plurality of wire guides 40 side-by-side, woven, or twisted together along a portion of the wire guides 40.

FIG. 5C also shows a self-expanding end portion 70 comprising a plurality of concave wire members 75 provide improved radial force and decreases trauma to the interior surface of the body lumen, which is preferred in circulatory applications, while helping to prevent the wire guide 40 from migrating within the body lumen. The self-expanding end portion 70 and other features shown in FIG. 5C may be used in an embodiment having a single wire guide 40, instead of the plurality of wire guides 40. In one embodiment of the concave self-expanding end portions 70, the concave wire members 75 have a somewhat cylindrical shape, potbelly stove shape, mushroom shape, and/or ribbed shape of an opened umbrella relative to a longitudinal axis 16 they bow outward intermediate the wire member distal end portion 70′ and wire member proximal end portion 70″ and/or the holding body mounting end portion 62. In one embodiment, the holding body mounting end portion 62 is a tubular hub that crimps the wire member proximal end portions 70″; the hub has a minimal diameter for the size of wire used to form the wire members 75. Thus, a self-expanding end portion 70 having a plurality of concave 76 wire members 75 closely models a cylinder, and cylindrical self-expanding end portions 70 are advantageous in assuming the natural configuration of a body lumen and forming an atraumatic, removable engagement against the inner wall of the body lumen. In one embodiment, the expanded radially outwardly diameter of the concave self-expanding end portion 70 is significantly oversized, which essentially stretches the inner wall of the body lumen to the point of a spring biased engagement between the periphery of the concave self-expanding end portion 70 and the inner wall of the body lumen. By giving the wire members 75 a concave 76 shape, the self-expanding end portion 70 provides an arcuate attachment to the inner wall of the body lumen to prevent wire guide 40 migration within the body lumen while providing a better fit to the curve of the inner wall of the body lumen, thereby leaving fewer and smaller gaps between the self-expanding end portions 70 and the vessel wall.

In an embodiment where the self-expanding end portion 70 comprises a plurality of convex 76′ wire members 75, the convex self-expanding end portion 70 provides improved tacking characteristics to prevent wire guide 40 migration within the body lumen. In one embodiment of the convex self-expanding end portions 70 fan out relative to the longitudinal axis 16, whereby the convex wire members 75 have a somewhat conical shape, tubular shape, hourglass shape, and/or skirt shape relative to the longitudinal axis 16 they bow inward intermediate the wire member distal end portion 70′ and wire member proximal end portion 70″ and/or the holding body mounting end portion 62. In one embodiment, the holding body mounting end portion 62 is a tubular hub that crimps the wire member proximal end portions 70″; the hub has a minimal diameter for the size of wire used to form the wire members 75. Thus, a self-expanding portion 70 having a plurality of convex 76′ wire members 75 provides discrete points of attachment to the inner wall of the body lumen to tack up and better hold the wire guide 40 in place and minimize migration. Thus, when a wire guide distal portion 46 is positioned within the body lumen and the self-expanding end portion 70 expandably deployed therein, the wire member distal end portion 70′ makes good contact with the inner wall of the body lumen.

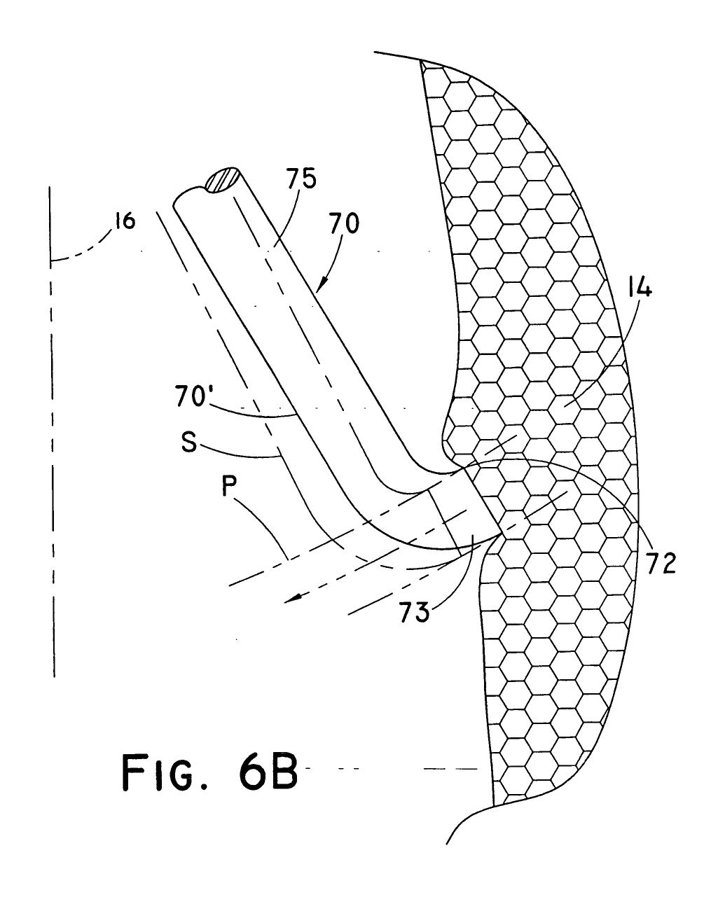

FIG. 6 shows a wire guide 40 having a distal portion 46 and a holding body 60 with a mounting end portion 62 operatively coupled to the wire guide distal portion 46, wherein the holding body 60 comprises a self-expanding end portion 70 having one or more anchoring members 72, 74, 74′. In one embodiment, the anchoring member 72 is a barb. FIG. 6 also shows that the anchoring member may comprise a wire member distal end portion 70′ without a barb, such as a wire member distal end portion 70′ of a convex 76′ wire member 75 (FIGS. 5A, 5D) or a concave 76 wire member 75 (FIGS. 5B, 5C), which as discussed above are configured in their radially expanded state to substantially prevent migration or the wire guide distal portion 46 within the body lumen. Generally stated, the self-expanding end portion 70 may have a plurality (e.g., two or more, or any combination of two or more) of anchoring members 72, 74, 74′, and/or wire member distal end portions 70′. Furthermore, the self-expanding end portion 70 of FIGS. 4A-4D, 6, and 6A-6D may be combined with self-expanding end portions shown schematically in FIGS. 7 through 11, and vice versa.

Anchoring members 72, 74, 74′ (including a wire member distal end portions 70′) may be any bend, arc, curved portion, J-shape, S-shape, T-shape, V-shape, X-shape, C-shape, Z-shape, shape, sickle-shaped, curved, bent, or hook device for anchoring the self-expanding end portion 70 to the inner wall of the body lumen when the physician withdraws the outer sheath 20 proximally. In one embodiment, a wire member 75 may have one or more anchoring members 72, 74, 74′ operatively coupled to or preferably integral with the wire member distal end portions 70′, wherein the mounting end portion 62 may be operatively coupled by any of the means discussed above for securing the mounting end portion 62 to the wire guide distal portion 46. The anchoring members 72, 74, 74′ (including a wire member distal end portions 70′) comprises a super-elastic memory metal alloy, such as nitinol, so as to not damage or harm the inner wall of the body lumen.

In addition to withdrawing the outer sheath 20, the physician optionally “pushes” the wire guide proximal portion 42 (shown in FIGS. 1 and 2) distally to ensure that the wire guide distal portion 46 (shown in FIGS. 1 and 2) remains at the desired target site and does not withdraw proximally with the withdrawal of the outer sheath 20. In other words, before deployment, the holding body 60 is constrained to a first radially compressed configuration within the outer sheath first end portion 22, and exits the outer sheath first end opening 24 as the outer sheath 20 is withdrawn. The self-expanding portion 70 comprises any super-elastic memory metal alloy described above, such as nitinol, that expands to a second radially expanded resilient configuration and, thereby, engage the inner wall of the body lumen in order to prevent migration of the wire guide distal portion 46. As will be understood, “pushing” on the wire guide proximal portion 42 counters the urge for the wire guide 40 to prolapse proximally with the withdrawing of the outer sheath 20 and will keep the wire guide distal portion 46 from translating proximally as a result of the outer sheath first end portion 22 being pulled over the self-expanding end portion 70; thereby “pushing” holds the wire guide distal portion 46 in place at the desired deployment site within the patient's body.

FIGS. 6A through 6D show enlarged views of embodiments of a wire member 75 of a self-expanding end portion 70. If FIG. 6A, the wire member 75 comprises an anchoring member 72 having a proximal anchoring hook 73, which anchoring member 72 may be implemented according to one aspect of the invention. In addition, FIGS. 6B, 6C, and 6D illustrate alternative embodiments of anchoring members 72, 74, 74′ comprising a super-elastic memory metal alloy, such as nitinol, that atraumatically engages a body lumen wall 14 so that the anchoring members 72, 74, 74′ may release the body lumen wall 14 without causing relatively serious tearing thereto.

The self-expanding end portion 70 of the wire member 75 according to these embodiments further comprises an anchoring hook 73, anchoring members 72, 74, 74′ distal to the anchoring hook 73, and a self-expanding end axis “S.” The “axis S” could be straight or curved, because the self-expanding end portion 70 is flexible. Along these lines, the self-expanding end portion 70 is configured to follow a path “P” between a first radially compressed configuration when constrained by the outer sheath and a second radially expanded resilient configuration for engaging the anchoring hook 73 and more particularly the anchoring members 72, 74, 74′ with the body lumen wall 14. As FIG. 6A illustrates (and also shown in FIGS. 6B, 6C, and 6D), the path “P” of the self-expanding end portion 70 and/or the wire member distal end portion 70′ falls between an angle a that measures from about 20 degrees and about 40 degrees from the longitudinal axis 16 and/or 20 to 40% deflection, which is possible with super-elastic memory metal such as nitinol. The anchoring hook 73 is curved away from a wire member distal end portion self-expanding axis “S” (hereafter, “self-expanding axis” or “self-expanding axis “S”) and terminates at one or more anchoring members 72, 74, 74′ that anchor to the body lumen wall 14 as shown in FIGS. 6B, 6C, and 6D.

As illustrated in FIG. 6B, the anchoring hook 73 and more particularly the anchoring member 72 is shown to atraumatically engage the body lumen wall 14. An anchoring hook 73 according to the invention comprises any suitable bend having an angle of up to about 90 degrees relative to the self-expanding axis “S.” In other words, the anchoring hook 73 is limited to about 90 degrees as measured against the self-expanding axis “S” as the reference. Otherwise stated, choosing the axis “S” as an abscissa in a plane Cartesian coordinate system, the anchoring hook 73 may assume an acute angle relative to the axis “S.”

In this embodiment, the anchoring hook 73 extends arcuately along the path P to a tangent point on the path P. The anchoring hook 73 and more particularly the anchoring member 72 will anchor in the body lumen wall 14 (not shown) when the self-expanding end portion 70 and/or wire member distal end portion 70′ is allowed to radially expand to a second configuration and engage the body lumen wall 14 to substantially keep the distal portion from moving relative to the inner wall 14 of the body lumen and from moving proximally relative to the proximal withdrawal of the outer sheath 20 (not shown).

Because the anchoring hook 73 is limited to about 90 degrees, the anchoring hook 73 may move in reverse along the path P and back to a first radially compressed configuration with minimal trauma and tearing to the body lumen wall 14. Thus, a physician may back-load the outer sheath 20 over the wire guide 40. In back-loading a wire guide 40, the physician inserts a distal end portion 22 of the outer sheath 20 over the proximal portion 42 of the wire guide 42, which is received within the outer sheath lumen 32. The outer distal end portion 22 and elongate flexible middle section 30 are advanced distally over the wire guide elongate flexible intermediate section 44, the wire guide distal portion 46, and the holding body 60 comprising the self-expanding end portion 70 to collapse the holding body self-expanding end portion 70 (i.e., for purposes of removing or repositioning the wire guide 40) so that the anchoring hook 73 or anchoring members 72, 74, 74′ (including a wire member distal end portions 70′) are constrained by the outer sheath distal first end portion 22 and thereby positioned at the surgical site within the body without ripping, tearing, or lacerating the body lumen wall 14. Of course, the holding body self-expanding end portion 70 may also be collapsed back to a first radially compressed configuration by withdrawing the wire guide through a working channel of a PTA device and/or a channel of an accessory device.

In FIGS. 6B, 6C, and 6D, the physician has withdrawn the outer sheath distal first end portion 22 proximally over the collapsed self-expanding end portion 70, thereby allowing the self-expanding end portion 70 to move from a first radially compressed configuration to a second radially expanded resilient configuration. As a result, the anchoring members 72, 74, 74′ in a second radially expanded resilient configuration thereby engage the body lumen wall 14. As discussed above, the anchoring members 72, 74, 74′ allow for atraumatic removal from, and without causing substantially any tearing to, the body lumen wall 14.

FIG. 6C further depicts a wire member 75 of a self-expanding end portion 70 comprising two anchoring members 74, 74′ for migration resistance, the an end face 72″ between the two anchoring members 74, 74′ that is recessed for migration resistance. The recessed end face 72″ is formed by any suitable method (grinding, milling, machining, molding, and the like, to name a few). In this embodiment, anchoring hook 73 extends to the anchoring member 74′, and an anchoring hook 73′ extends to the anchoring member 74. The anchoring hooks 73, 73′ move arcuately along the path P to a tangent point on the path P. The anchoring hook 73′ has an angle of up to about 90 degrees relative to the self-expanding axis “S,” while the anchoring hook 73 forms an acute angle relative to the self-expanding axis “S.”

FIG. 6D depicts another embodiment of a wire member 75 of a self-expanding end portion 70 having an anchoring member 72 engaging a body lumen wall 14 according to the invention. As shown, extending the length of the anchoring hook 73 increases the depth to which the anchoring hook 73 and more particularly the anchoring member 72 atraumatically engage the body lumen wall 14. As with the other embodiments, the anchoring hook 73 moves arcuately along the path P to a tangent point on the path P. The anchoring hook 73 extends to the anchoring member end face 72′, and an anchoring hook 73′ extends to the anchoring member 72 at an angle of up to about 90 degrees relative to the self-expanding axis “S.”

FIG. 7 shows an embodiment of wire members 80, 82 comprising a plurality of loops. More particularly, a wire guide 40 according to FIG. 7 comprises a distal portion 46 and a holding body 60 with a mounting end portion 62 operatively coupled to the wire guide distal portion 46, wherein the holding body 60 comprises a self-expanding end portion 70 that comprises a plurality of wire members 80, 82 forming loops. Before deployment, the holding body 60 is constrained to a first radially compressed configuration within the outer sheath first end portion 22, and exits the outer sheath first end opening 24 as the outer sheath 20 is withdrawn. When expanded to a second radially expanded resilient configuration, the plurality of looped wire members 80, 82 form an incandescent light bulb or “basket” shape and/or “bulb” shape. Baskets are known in the retrieval art and are commonly used to remove an object, such as a stone or other undesirable object, form a body cavity. Examples of baskets are described in U.S. Pat. No. 5,725,552, the disclosure of which is incorporated herein in its entirety, in the retrieval art and are commonly used to remove an object, such as a stone or other undesirable object, form a body cavity. Examples of collapsible baskets are disclosed in U.S. Pat. No. 7,001,409, the disclosure of which is incorporated herein in its entirety. Examples of expandable intraluminal devices and methods of deploying such devices are described in U.S. Patent Application, Publication No. 2004/0225322A1 and having an application Ser. No. 10/804,386, the disclosure of which is incorporated herein in its entirety. Baskets have not been previously used, however, in connection with an improved wire guide having distal anchoring devices designed specifically to hold the wire guide 20 to the inner wall of the body lumen to prevent migration of the wire guide 40. To the contrary, conventional wire guides are wires intended to be temporarily located within a body lumen such as an artery, for example, for placement of a catheter to a certain location in the body, but the conventional wire guide itself has a minimal profile terminating at a ball-like head that lacks structure for placement without undesirable migration.

The embodiment of the basket illustrated in FIG. 7 comprises a plurality of wire members 80, 82 forming loops, which wire members 80, 82 comprise super-elastic memory metal alloys, such as nitinol, that are woven or braided into a tubular configuration and then heat set in a mold in a manner described in U.S. Pat. No. 6,123,715 to Curtis Amplatz, the contents of which are hereby incorporated by reference. Furthermore, the plurality of loops 80, 82 are joined at a mounting end portion 62 secured to the wire guide distal portion 46 and slideably constrained by the outer sheath 20. A plurality of loops 80, 82 are self-expanded to the shape of a basket for percutaneous transluminal angioplasty according to this alternative embodiment of the invention of the invention and may be made from tubular mesh which can be compressed for delivery through catheter but which, on delivery to the target site, expands into a “flat” or “disc-like” shape appropriate for sealing a puncture site. Accordingly, the plurality of loops 80, 82 has an expanded configuration and a collapsed configuration.

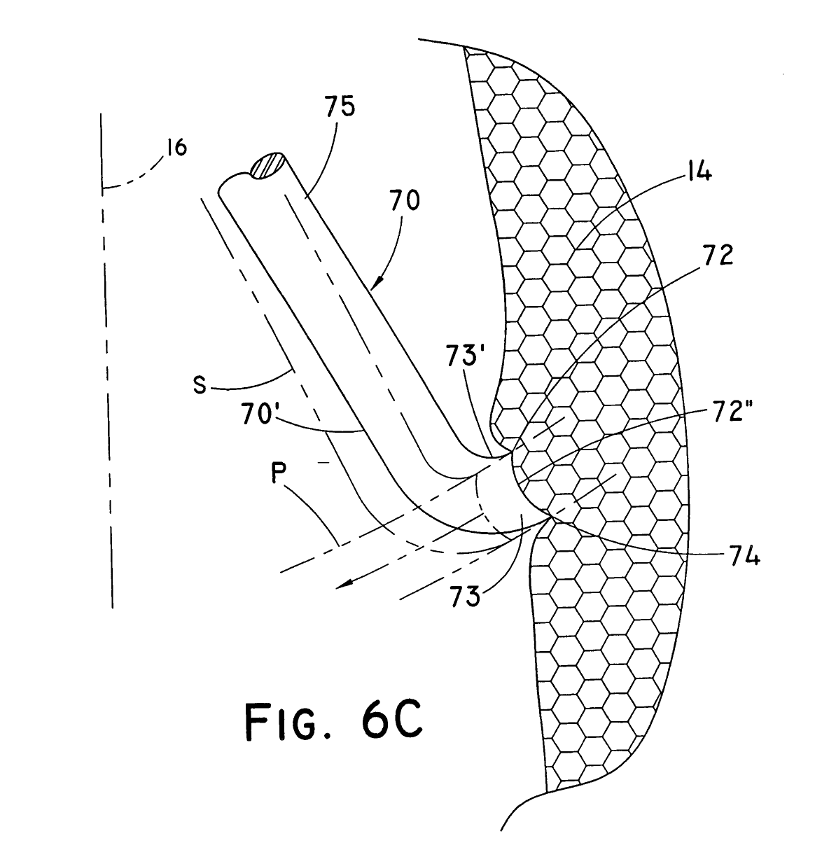

FIG. 8 shows another alternative embodiment of a wire guide 40 having a distal portion 46 and a holding body 60 with a mounting end portion 62 operatively coupled to the wire guide distal portion 46, wherein the holding body 60 comprises a self-expanding end portion 70 that, when expanded to a second radially expanded resilient configuration, roughly resembles a tri-leaflet, bulb-like, or “flame” configuration. Before deployment, the holding body 60 is constrained to a first radially compressed configuration within the outer sheath first end portion 22, and exits the outer sheath first end opening 24 as the outer sheath 20 is withdrawn, thereby assuming a second radially expanded resilient configuration for engaging the inner wall of the body lumen and preventing migration of the wire guide distal portion 46. This embodiment of the holding body 60 further has a self-expanding end portion 70 comprising a plurality of wire members 75 that terminate distally at a tether 84 by any suitable means, such as crimping, twisting, tying, welding, soldering, brazing, gluing, adhesives, heat bonding, glue, adhesives, resins, passive oxide layer covering, aluminum paste flux, and/or combination thereof. Securing the self-expanding end portion 70 at a tether 84 helps to keep the ends substantially aligned with the longitudinal axis of the wire guide distal portion 46. A further advantage is that a closed round end is provided to the holding body 60. Another advantage is for shaping the self-expanding end portion 70 into a desired configuration, such as a bent tip, and in customizing the axial and lateral dimensions, such as when a short squatty body is desirable. The tether 84 is approximately aligned with the longitudinal axis 16 so as to provide another benefit: as the outer sheath 20 withdraws proximally, the self-expanding end portion 70 of the holding body 60 expands approximately from the center of the passageway outward.

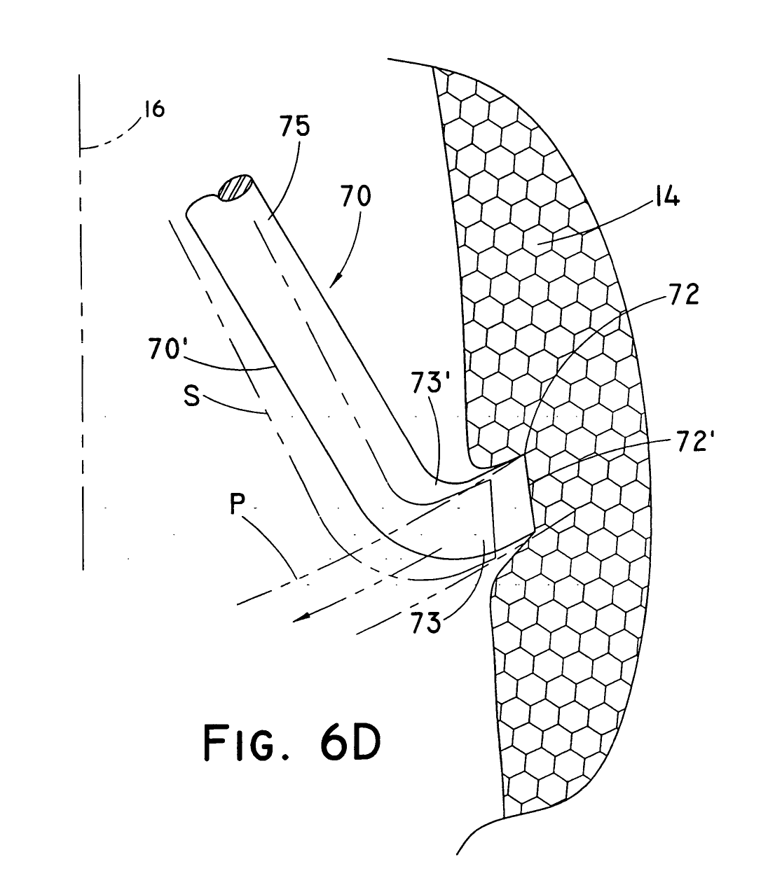

FIG. 9 shows another alternative embodiment of wire members 86 comprising a plurality of loops. More particularly, a wire guide 40 according to FIG. 9 comprises a distal portion 46 and a holding body 60 with a mounting end portion 62 operatively coupled to the wire guide distal portion 46, wherein the holding body 60 comprises a self-expanding end portion 70 that comprises a plurality of wire members 86 each forming an interwoven loop closed at the mounting end portion 62. The interwoven looped wire members 86 form a knot-like or intertwined configuration that provides advantages of shortening the axial expansion while achieving an optimal radial expansion of the self-expanding end portion 70. Before deployment, the holding body 60 is constrained to a first radially configuration within the outer sheath first end portion 22, and exits the outer sheath first end opening 24 as the outer sheath 20 is withdrawn. The self-expanding portion 70 comprises any super-elastic memory metal alloy described above, such as nitinol, that expands to a second radially expanded resilient configuration and, thereby, engage the inner wall of the body lumen in order to prevent migration of the wire guide distal portion 46.

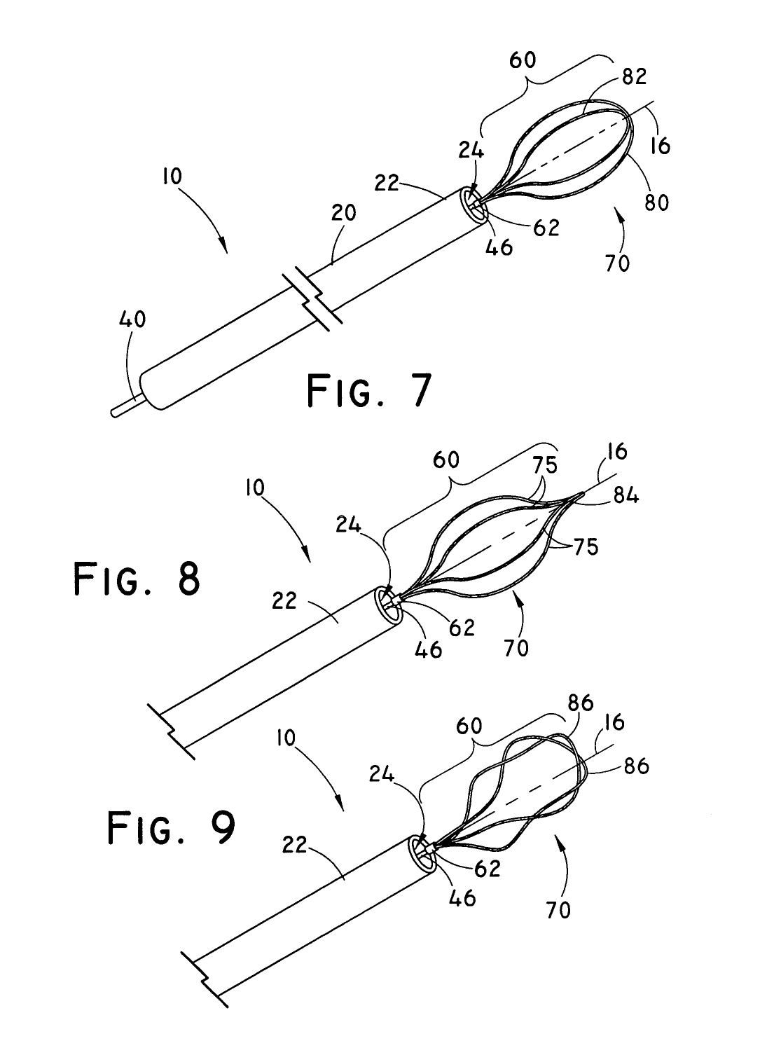

FIG. 10, in yet another embodiment, shows another alternative embodiment of a holding body 60 having a mounting end portion 62 and a self-expanding end portion 70. A wire member 88, which may be one wire or a plurality of wires positioned substantially side-by-side or weaved or braided together, forms a corkscrew. This provides an advantage by increasing the surface area of the wire member 88 that engages the inner wall of the body lumen. In other words, numerous turns may push against the inner wall of the body lumen and help to hold the wire guide 40 in place. In addition, the tapered shape provides a gradual decrease in the diameter by which the inner wall of the body lumen is pushed radially for engagement. Moreover, as the outer sheath 20 withdraws proximally, the wire member 88 expands from a hub 89 approximately aligned with the longitudinal axis 16 such that the wire member expands roughly at the center of the body lumen, so that the expansion engages all “sides” of the inner wall of the body lumen substantially equally.

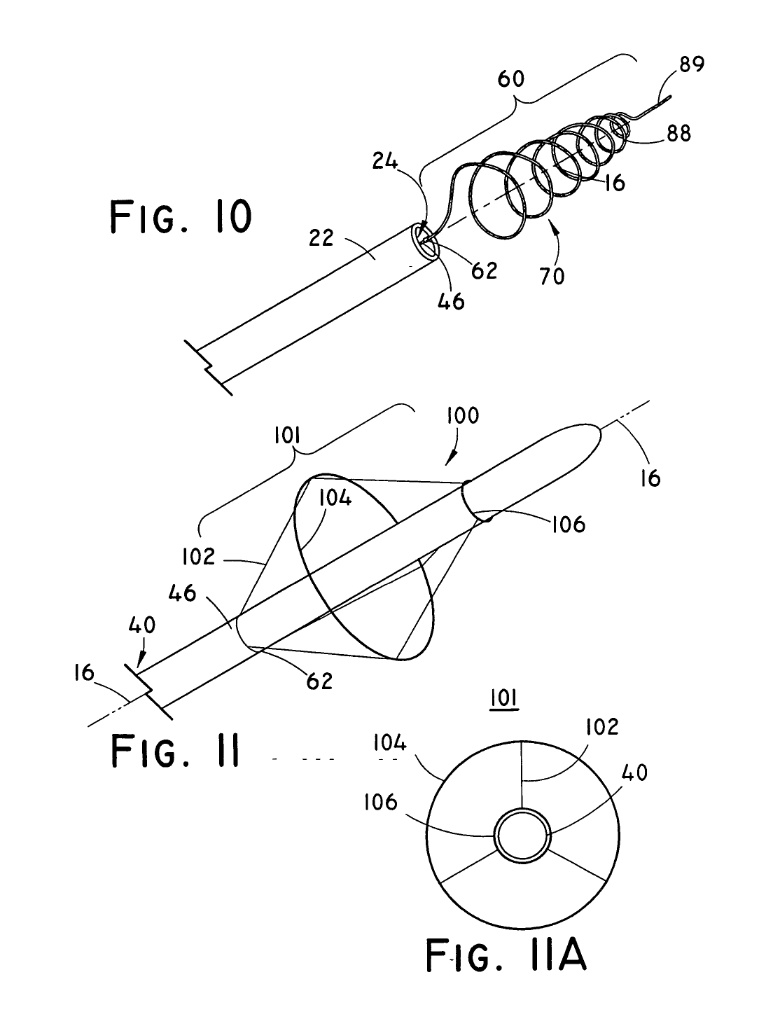

As shown in FIG. 11, this invention also pertains to a wire guide 40 having a distal portion 46, and a wire guide anchor device 100 having a mounting end portion 62 operatively coupled to the wire guide distal portion 46, the wire guide anchor device 100 further comprising a distal self-expanding suspension portion 101, wherein the distal self-expanding suspension portion 101 is self-expanding and capable of assuming a first radially compressed configuration when constrained within an outer sheath 20 and a second radially expanded resilient configuration when the 20 outer sheath is withdrawn proximally relative to the wire guide anchor device 100. Fully expanded, the wire guide anchor device 100 is approximately from 0.5 cm to about 1.5 cm in length along the longitudinal axis 16, and in one embodiment is about 1.0 cm in length along the longitudinal axis 16, although the length may be greater or lesser than this range depending on the size of the intended body lumen. The wire guide anchor device 100 may have any suitable outer diameter depending on the size of the intended vessel passageway, and in one embodiment the wire guide anchor device 100 is approximately 0.2 cm to approximately 1.0 cm in diameter when fully expanded, although the diameter may be greater or lesser than this range depending on the size of the intended body lumen.

The distal self-expanding suspension portion 101 comprises a plurality of stabilizers 102 with an outer ring 104 disposed about the stabilizers 102. In one embodiment, there are at least three or more stabilizers 102 and preferably at least five or more stabilizers 102, because the greater number more accurately assume an arc-shaped cylindrical self-expanding end portion 70 when expanded to a second radially expanded resilient configuration. The stabilizers 102 are operatively coupled to the mounting end portion 62 by any suitable means, such as crimping, tying, welding, soldering, brazing, gluing, adhesives, heat bonding, glue, adhesives, resins, passive oxide layer covering, aluminum paste flux, and/or combination thereof. In one embodiment, the stabilizers 102 are secured to the outer ring 104 by any suitable means so as to prevent the outer ring 104 from moving distally into an abutting position with the hub 106 during deployment, although in an alternative embodiment the outer ring 104 is not secured to the stabilizers 102 and slides to a position intermediate the hub 106 and the mounting end portion 62 when the stabilizers 102 expand from to a second radially expanded resilient configuration. In another embodiment, the outer ring 104 would be fixed to the stabilizer 102 by wires, suture, and/or material such as Dacron, ePTFE Teflon, and the like. The stabilizers 102 are operatively coupled distally to a distal hub 106 by any suitable means, such as crimping, tying, welding, soldering, brazing, gluing, adhesives, and/or combination thereof.

In a preferred embodiment, the stabilizers 102 comprise any super-elastic memory metal alloy described above, such as nitinol, wherein the stabilizers expand to a second radially expanded resilient configuration and, thereby, engage the inner wall of the body lumen in order to prevent migration of the wire guide distal portion 46. This embodiment facilitates collapsing the wire guide anchor device 100 sliding the outer sheath first end portion over the stabilizers 102, thereby moving the hub 106 distally and the stabilizers 102 inward toward the longitudinal axis 16 and the wire guide anchor device 100 to a smaller diameter sized to fit within the outer sheath lumen. This does not preclude the possibility that the wire guide anchor device 100 have one or more stabilizers 102 made from material other than super-elastic memory metal alloy, such as thread by way of example and not by way of limitation.

In an alternative embodiment, however, the outer ring 104 may comprise a super-elastic memory metal alloy described above, such as nitinol, wherein the outer ring 104 expands to a second radially expanded resilient configuration and, thereby, engage the inner wall of the body lumen in order to prevent migration of the wire guide distal portion 46. In yet another embodiment, the stabilizers 102 as well as the outer ring 104 comprise super-elastic memory metal alloy. In one embodiment, the outer ring 104 is in the form of a self expanding stent that is resiliently compressed into a collapsible to a first, smaller diameter and due to its construction and material properties expandable to a second, larger diameter upon deployment. In its expanded configuration, the stent exhibits sufficient stiffness so that it will remain substantially expanded and exert a radially outward force in the vessel passageway on an inner wall of the body lumen. One particularly useful self-expanding stent is the Z-stent, introduced by Cook Incorporated, due to its ease of manufacturing, high radial force, and self-expanding properties. Examples of the Z-stent are found in U.S. Pat. Nos. 4,580,568; 5,035,706; 5,282,824; 5,507,771; and 5,720,776, the disclosures of which are incorporated in their entirety. The Zilver stent, introduced by Cook Incorporated, is another particularly useful self-expanding stent due to its nitinol platform and use of the Z-stent design properties. Examples of the Zilver stent are found in U.S. Pat. Nos. 6,743,252 and 6,299,635, the disclosures of which are incorporated in their entirety. The outer ring 104 may have an outer diameter slightly larger than the inner diameter of the body lumen. In the case of the renal, for example, the outer ring 104 would range anywhere from about 5 mm to about 9 mm in outer diameter, although the outer ring 104 could also be made in other sizes for applications in different body lumens.

In one embodiment, the hub 106 is sized to be slidably disposed about the wire guide distal portion 46 such that inserting the sheath distal end portion over the wire guide anchor device 100 moves the hub 106 distally along the longitudinal axis 16 and the wire guide distal portion 46, thereby allows collapsing the stabilizers 102 and outer ring 104 within the outer sheath lumen. In an alternative embodiment, the hub 106 is stationary, and the collapsing of the stabilizers 102 and outer ring 104 within the outer sheath lumen results from an actuator such as an auxiliary wire attached to the mounting end portion 62 in order to pull the stablizers 102 proximally and thereby collapse the stabilizers 102 and the outer ring 104 like closing an umbrella.

Before deployment, the stabilizers 102 and the outer ring 104 are constrained to a first radially configuration within the outer sheath first end portion, and exits the outer sheath first end opening as the outer sheath is withdrawn. In one embodiment, the outer sheath first end opening is slightly conically shaped so as to facilitate re-entry of the wire guide anchor device 100 within the outer sheath lumen at the outer sheath first end portion.

FIG. 11A is an end view of FIG. 11 taken along the line A-A. In addition to holding the wire guide 40 substantially in a centered position within the body lumen, the outer ring 104 of the wire guide anchor device 100 helps to may the stabilizers 102 atraumatic against the inner wall of the body lumen. The hub 106 encircles the distal portion of the wire guide 40 at a central location of the wire guide anchor device. The hub 106 is shown spaced from the wire guide 40, thereby making the hub 106 slideable relative to the wire guide 40. Stabilizers 102 extend radially from the hub 106 to points at the edge of the outer ring 104. The stabilizers 102 extend proximally from the outer ring 104 to the mounting end portion 62 (FIG. 11). The stabilizers 102 are disposed about the longitudinal axis 16 such that the stabilizers 102 and/or the outer ring 104 are capable of engaging opposing inner walls of the body lumen and thereby preventing the wire guide 40 from migrating within the body lumen.

In one embodiment, the outer ring 104 expands substantially perpendicular to the longitudinal axis 16 of the wire guide 40 at or near the central axis of the ring 104. The stabilizers 102 and outer ring 104 are shown in a second radially expanded resilient configuration, and the hub 106 disposed about the wire guide 40. Once the stabilizers 102 oppose the inner wall of the body lumen distal to a lesion, for example, the stabilizers 102 optionally suspend the wire guide distal portion 46 (FIG. 11) approximately centrally located within the body lumen and hold the wire guide distal portion 46 (FIG. 11) from advancing or pulling out.

As will be understood by one of ordinary skill in the art, the embodiments may be combined. For example, a self-expanding end portion 70 according to FIGS. 1, 2, 4, 5A-5D, 6, and 7 may have a plurality of wire members 75 with wire member distal end portions 70′ operatively coupled to a distal hub 106 (FIGS. 11, 11A) by any suitable means, such as crimping, tying, welding, soldering, brazing, gluing, adhesives, and/or combination thereof. In addition, the plurality of wire members 75 in FIG. 8 may terminate distally at a distal hub 106 (FIGS. 11, 11A) instead of a tether 84 (FIG. 8), where terminate includes the wire members 75 being operatively coupled to the distal hub 106.

Methods

Methods of providing a providing a wire guide anchoring system that is migration resistant to an inner wall of a body lumen of a patient for percutaneous procedures are also provided. The embodiments use an outer sheath 20 to deliver a wire guide 40 having a distal portion 46 operatively coupled to a self-expanding anchor device 70, 101 (e.g., a wire guide self-expanding distal portion 70 of a holding body 60 as shown and described relating to FIGS. 1-2, 4, 5A-5D, 6, 6A-6D, 7-10, or a distal self-expanding suspension portion 101 of a wire guide anchor device 100 as shown and described relating to FIGS. 11 and 11A) (individually and collectively, “self-expanding anchor device 70, 101 ”) comprising super-elastic memory metal alloy that is capable of assuming a first radially compressed configuration when constrained by the outer sheath and a second radially expanded resilient configuration that engages an inner wall of a body lumen when the sheath is withdrawn proximally relative to the self-expanding anchor device 70, 101 for anchoring the wire guide distal portion 46 to a desired position during a medical procedure.

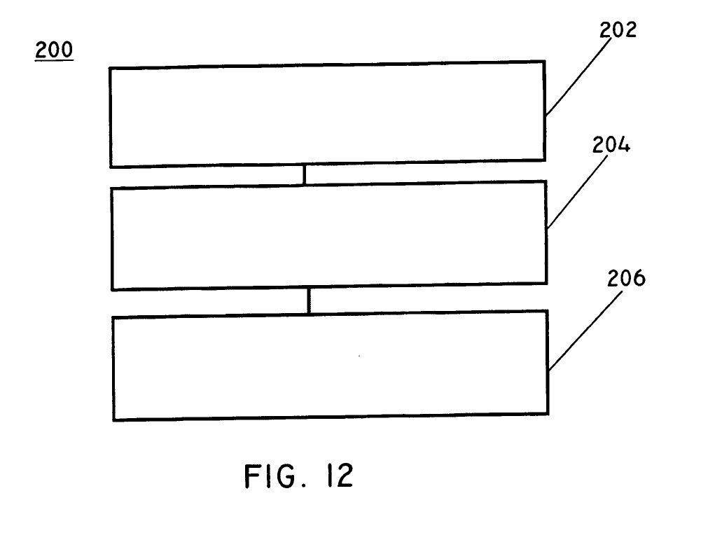

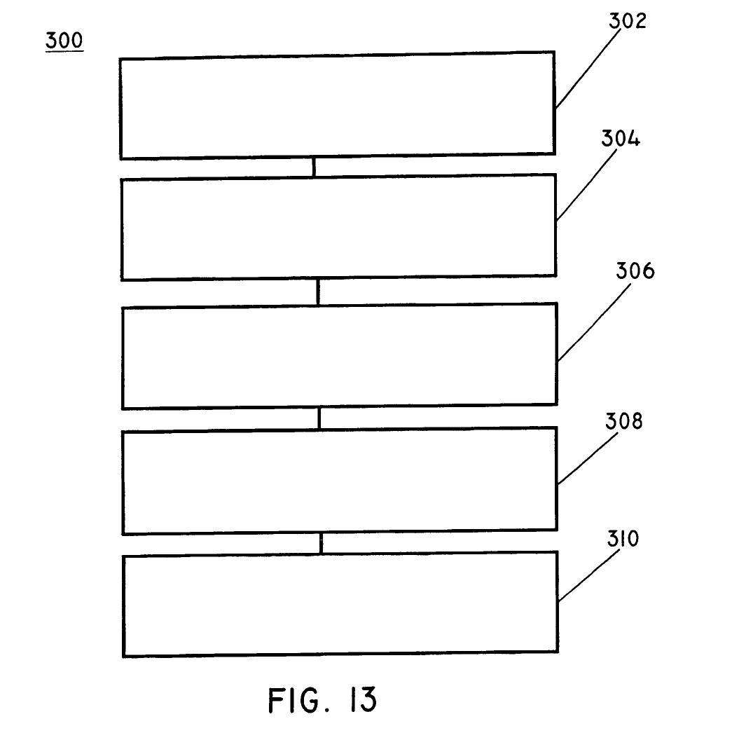

According to one embodiment of the method, a wire guide anchoring system having an outer sheath 20 with a distal first end portion 22, a proximal end portion 26, and an elongate flexible middle section 30, the outer sheath further having a first opening 24 and a second opening 28 defining a lumen 32 therebetween, the lumen 32 being sized to slideably receive a wire guide 40 comprising a wire guide proximal portion 42, a wire guide elongate flexible intermediate section 44, and a wire guide distal portion 46 operatively coupled to self-expanding anchor device 70, 101 comprising super-elastic memory metal alloy that is capable of assuming a first radially compressed configuration when constrained by the outer sheath and a second radially expanded resilient configuration that engages an inner wall of a body lumen when the outer sheath 20 is withdrawn proximally relative to the self-expanding anchor device 70, 101. The outer sheath first end portion 22 is positioned at a target site in said patient with the self-expanding anchor device 70, 101 within the outer sheath lumen 32 and in first radially compressed configuration. The outer sheath 20 is withdrawn proximally from the self-expanding anchor device 70, 101 such that the self-expanding anchor device 70, 101 radially expands to the second radially expanded resilient configuration and engages the inner wall of the body lumen, the self-expanding anchor device 70, 101 in the second radially expanded resilient configuration being capable of substantially anchoring the wire guide distal portion to said inner wall of said body lumen. This method is shown discussed more particularly relative to FIG. 12 for self-expanding anchor device 70 (e.g., FIGS. 1-2, 4, 5A-5D, 6, 6A-6D, 7-10) and FIG. 13 for a self-expanding anchor device 101 (e.g., FIGS. 11 and 11A).

FIG. 12 shows one embodiment of a method 200. In this method, an improved wire guide anchoring system is provided (step 202) having an outer sheath 20 with a distal first end portion 22, a proximal second end portion 26, and an elongate flexible middle section 30, the outer sheath 20 further having a first opening 24 and a second opening 28 defining a lumen 32 therebetween, the lumen 32 being sized to slideably receive a wire guide 40 comprising a proximal portion 42, an elongate flexible intermediate section 44, and a distal portion 46 that operatively couples with a mounting end portion 62 of a holding body 60 having a self-expanding end portion 70 comprising super-elastic memory metal alloy capable of assuming a first radially compressed configuration when constrained by the outer sheath and a second radially expanded resilient configuration when the sheath is withdrawn proximally relative to the wire guide distal portion. The outer sheath distal end 22 having the wire guide distal portion 46 disposed within the outer sheath lumen 32 and self-expanding end portion 70 of the holding body 60 held in the first radially compressed configuration is positioned (step 204) at a target site in said patient. The outer sheath 20 is then withdrawn (step 206) from the wire guide distal portion 46 so that the self-expanding end portion 70 of the holding body 60 is allowed to expand to the second radially expanded resilient configuration and engage the inner wall of a body lumen to substantially keep the wire guide distal portion 46 from moving relative to the proximal withdrawal of the outer sheath 20.

FIG. 13 is a block diagram showing an alternative embodiment of method 300 for using an improved wire guide anchoring system. An elongate outer sheath 20 is provided (step 302), the sheath having first and second openings 24, 26 defining a lumen 32 therebetween and sized to slideably receive an elongate inner wire guide 40. A wire guide 40 is provided (step 304), the wire guide 40 having a proximal portion 42, an elongate flexible intermediate section 44, and a distal portion 46 that secures a proximal mounting end portion 62 of a wire guide anchor device 100 that comprises a distal self-expanding suspension portion 101 comprising a plurality of stabilizers 102 operatively coupled to the proximal mounting end portion 62 and distally to a distal hub 106 and having an outer ring 104 positioned intermediate the mounting end portion and the hub 106, wherein the distal self-expanding suspension portion 101 comprises super-elastic memory metal alloy and is capable of assuming a first radially compressed configuration when constrained by the outer sheath 20 and a second radially expanded resilient configuration when the outer sheath 20 is withdrawn proximally relative to the wire guide distal portion 46 so as to not be constrained by the outer sheath. A portion of the wire guide 40 is received (step 306) within the sheath lumen 32 such that the distal self-expanding suspension portion 101 is compressed into a first radially compressed configuration. The outer sheath distal end 22, with the wire guide distal portion 46 within the outer sheath lumen 32 and the distal self-expanding suspension portion 101 in a first radially compressed configuration within the outer sheath lumen 32, is inserted distally (step 308) into a patient percutaneously and the wire guide distal portion 46 is subsequently positioned at or near a target site. The outer sheath 20 is withdrawn (step 310) from the wire guide distal portion 46 such that the distal self-expanding suspension portion 101 expands to the second radially expanded resilient configuration and stabilizers 102 and/or outer ring 104 engage the inner wall of a body lumen sufficient to substantially keep the wire guide distal portion 46 from moving proximally relative to the proximal withdrawal of the outer sheath 20.

A method of using an improved wire guide anchoring system for holding a wire guide in place need not be performed sequentially. For instance, in method 300, a wire guide 40 may be provided (step 304) before an outer sheath 20 is provided (step 304). Also, an outer sheath 20 may be inserted (step 308) distally to a target site, and then the wire guide 40 received (step 306) into the outer sheath 20 by a back-loading procedure whereby the physician inserts a distal first end portion 22 of the outer sheath 20 over the proximal end 42 of the wire guide 40 and slides the outer sheath first end portion 22 over the distal self-expanding suspension portion 101.

After the self-expanding end portion 70 of the holding body 60 (or the wire guide anchor device 100) is radially expanded to a second configuration to engage the inner wall of a vessel passageway sufficient to substantially keep the distal portion from moving proximally relative to the proximal withdrawal of the outer sheath 20, the wire guide 40 may be removed in many ways that collapse the self expanding end portion 70 (or the wire guide anchor device 100) may back to first radially compressed configuration. For instance, the outer sheath distal first end opening 22 may be disposed about the proximal portion 42 of the wire guide 40, thereby receiving the wire guide 40 in the outer sheath lumen 32, and the outer sheath distal end portion 22 moved distally over the self-expanding end portion 70 of the holding body 60 (or the wire guide anchor device 100). Alternatively, the wire guide 40 may be withdrawn through a working channel of a PTA device. Also, the holding body self-expanding end portion 70 (or the stabilizers 102 of the wire guide anchor device 100) may be cooled to transform the material to martensite, which is more ductile than austenite, making the self-expanding end portion 70 (or the stabilizers 102 of the wire guide anchor device 100) more malleable and more easily collapsible. In one embodiment, the outer sheath first end opening is slightly conically shaped so as to facilitate re-entry of the wire guide anchor device 100 and/or the holding body 60 within the outer sheath lumen at the outer sheath first end portion.

It is intended that the foregoing detailed description of the medical devices and methods be regarded as illustrative rather than limiting, and that it be understood that it is the following claims, including all equivalents, that are intended to define the spirit and scope of this invention. Terms are to be given their reasonable plain and ordinary meaning. Also, the embodiment of any figure and features thereof may be combined with the embodiments depicted in other figures. Other features known in the art and not inconsistent with the structure and function of the present invention may be added to the embodiments.

While particular elements, embodiments and applications of the present invention have been shown and described, it will be understood, of course, that the invention is not limited thereto since modifications may be made by those skilled in the art, particularly in light of the foregoing teachings. Therefore, it is therefore contemplated by the appended claims to cover such modifications as incorporate those features which come within the spirit and scope of the invention.

What is claimed is:

1. A wire guide device for deployment in a body lumen through an outer sheath comprising:
a wire guide having a proximal portion, an elongate flexible intermediate section, and a distal portion; and
a holding body comprising a self-expanding end portion, a mounting end portion, and a longitudinal axis, the mounting end portion operatively coupling the self-expanding end portion to the wire guide distal portion, the self-expanding end portion being capable of assuming a first radially compressed configuration when constrained by said outer sheath and a second radially expanded resilient configuration disposed about the longitudinal axis and that engages said body lumen wall when said outer sheath is withdrawn proximally relative to the holding body.

2. The device of claim 1 wherein the self-expanding end portion comprises a plurality of wire members comprising super-elastic memory metal alloy selected from the group consisting of copper-zinc-aluminum, copper-aluminum-nickel, iron-manganese-silicon, gold-cadmium, copper-aluminum, copper-aluminum-nickel, cobalt-chromium-nickel-molybdenum-iron alloy, cobalt-chrome alloy, titanium, thermosetting polymers, thermoplastic polymers, nickel-titanium alloy, and any combination thereof.

3. The device of claim 2 wherein wire members each comprise a proximal end portion and a distal end portion, the proximal end portion being operatively coupled to the holding body mounting end portion.

4. The device of claim 3 wherein the wire member distal end portion is configured to move along a path between the first radially compressed and the second radially expanded resilient configuration, the path being at least a 20 degrees deflection from the longitudinal axis.

5. The device of claim 4 wherein the wire member distal end portion further comprises an anchoring hook that is curved away from a wire member distal end portion self-expanding axis and terminates at an anchoring member.

6. The device of claim 5 further comprising a second anchoring hook that is curved away from the wire member distal end portion self-expanding axis and terminates at a second anchoring member, the two anchoring members forming a recessed end face therebetween for migration resistance.

7. The device of claim 3 wherein the wire member arcs radially away from the longitudinal axis, and the wire member proximal end portion curves radially toward the longitudinal axis.

8. The device of claim 3 wherein adjacent wire member distal end portions are joined end-to-end at an arcuate connection.

9. The device of claim 8 wherein the wire member comprises a concave arc relative to the longitudinal axis intermediate the wire member distal and proximal end portions in the second radially expanded resilient configuration.

10. The device of claim 8 wherein the wire member comprises a convex arc relative to the longitudinal axis intermediate the wire member distal and proximal end portions in the second radially expanded resilient configuration.

11. The device of claim 2 wherein the wire members comprise a plurality of loops.

12. The device of claim 2 wherein the wire members terminate distally at a tether.

13. The device of claim 2 wherein the wire members terminate distally at a hub.

14. A wire guide device for deployment in a body lumen comprising:
a wire guide having a proximal portion, an elongate flexible intermediate section, and a distal portion;
a wire guide anchor device having a proximal mounting end portion and a distal self-expanding suspension portion, the mounting end portion operatively coupled to the wire guide distal portion; and
an outer sheath having a distal first end portion, a proximal second end portion, and an elongate flexible middle section, the outer sheath further having a first opening and a second opening defining a lumen therebetween, the sheath lumen being sized to slideably receive the wire guide anchor device,
wherein the distal self-expanding suspension portion being self-expanding and being capable of assuming a first radially compressed configuration when constrained within the outer sheath and a second radially expanded resilient configuration when the outer sheath is withdrawn proximally relative to the wire guide anchor device.

15. The device of claim 14 wherein the self-expanding suspension portion comprises a plurality of self-expanding stabilizers comprising super-elastic memory metal alloy selected from the group consisting of copper-zinc-aluminum, copper-aluminum-nickel, iron-manganese-silicon, gold-cadmium, copper-aluminum, copper-aluminum-nickel, cobalt-chromium-nickel-molybdenum-iron alloy, cobalt-chrome alloy, titanium, thermosetting polymers, thermoplastic polymers, nickel-titanium alloy, and any combination thereof.

16. The device of claim 15 wherein the self-expanding stabilizers are operatively coupled distally to a distal hub.

17. The device of claim 16 wherein the hub is sized to be slidably disposed about the wire guide distal portion.

18. The device of claim 16 further comprising an outer ring disposed about the stabilizers intermediate the proximal mounting end portion and the distal hub.

19. The device of claim 18 wherein the outer ring is capable of assuming a first radially compressed configuration when constrained within the outer sheath and a second radially expanded resilient configuration when the outer sheath is withdrawn proximally relative to the wire guide anchor device.

20. The device of claim 18 wherein the outer ring comprises super-elastic memory metal alloy selected from the group consisting of copper-zinc-aluminum, copper-aluminum-nickel, iron-manganese-silicon, gold-cadmium, copper-aluminum, copper-aluminum-nickel, cobalt-chromium-nickel-molybdenum-iron alloy, cobalt-chrome alloy, titanium, thermosetting polymers, thermoplastic polymers, nickel-titanium alloy, and any combination thereof.

21. A method of providing a wire guide anchoring system that is migration resistant to an inner wall of a body lumen at a target site of a patient for percutaneous procedures, comprising:
providing a wire guide anchoring system having an outer sheath with a distal first end portion, a proximal end portion, and an elongate flexible middle section, the outer sheath further having a first opening and a second opening defining a lumen therebetween, the lumen being sized to slideably receive a wire guide comprising a wire guide proximal portion, a wire guide elongate flexible intermediate section, and a wire guide distal portion operatively coupled to self-expanding anchor device comprising super-elastic memory metal alloy that is capable of assuming a first radially compressed configuration when constrained by the outer sheath and a second radially expanded resilient configuration that engages said inner wall of said body lumen when the outer sheath is withdrawn proximally relative to the self-expanding anchor device;
positioning the outer sheath first end portion at said target site in said patient with the self-expanding anchor device within the outer sheath lumen and in the first radially compressed configuration;
withdrawing the outer sheath proximally from the self-expanding anchor device such that the self-expanding anchor device radially expands to the second radially expanded resilient configuration and engages the inner wall of a body lumen, the self-expanding anchor device in the second radially expanded resilient configuration being capable of substantially anchoring the wire guide distal portion to said inner wall of said body lumen.

22. The method of claim 21 further comprising inserting the outer sheath over the self-expanding anchor device and collapsing the self-expanding anchor device to the first radially compressed configuration.

23. The method of claim 22 further comprising withdrawing the outer sheath and the wire guide from said patient.
